(19) United States
(12) Patent Application Publication
(10) Pub. No.: US 2025/0372237 A1
(43) Pub. Date: Dec. 4, 2025

(54) METHODS AND SYSTEMS FOR GENERATING AND VISUALIZING PATIENT INFORMATION INCLUDING TUMOR HETEROGENEITY, THERAPY EFFECTIVENESS, AND SURVIVAL STATISTICS

(71) Applicant: KONINKLIJKE PHILIPS N.V., EINDHOVEN (NL)

(72) Inventors: YEE HIM CHEUNG, Boston, MA (US); Piotr Kaczmarek, Milton, MA (US); Jie Wu, Cambridge, MA (US)

(21) Appl. No.: 18/876,750

(22) PCT Filed: Jun. 27, 2023
(86) PCT No.: PCT/EP2023/067381

§ 371 Date: Dec. 19, 2024

Related U.S. Application Data
(60) Provisional application No. 63/357,018, filed on Jun. 30, 2022.
(60) Provisional application No. 63/449,325, filed on Mar. 2, 2023.

(51) Int. Cl. G16H 30/40; G16H 10/60; G16H 20/00; G16H 50/30; G16H 50/70

(52) U.S. Cl. CPC G16H 30/40; G16H 10/60; G16H 20/00; G16H 50/30; G16H 50/70

(57) ABSTRACT

A method (100) for visualizing heterogeneity of a patient's tumor, comprising: (i) receiving (120) information about a patient, comprising demographic and clinical information about the patient, an identification of the patient's tumor type, and a heterogeneity assessment of the patient's tumor; (ii) obtaining (130), based on the identification of the patient's tumor type and the heterogeneity assessment of the patient's tumor, therapy effectiveness information for each of the two or more subclones of the tumor: (iii) generating (140) a visual representation of the heterogeneity assessment of the patient's tumor and the obtained therapy effectiveness information for each of the two or more subclones of the tumor; and (iv) providing (150), via a user interface of the system, the generated visual representation, wherein the visual representation comprises a tumor heterogeneity graph and a therapy effectiveness graph.

### FIELD OF THE DISCLOSURE

The present disclosure is directed generally to methods and systems for generating and providing visual representations of patient information including tumor heterogeneity, therapy effectiveness, and survival statistics.

### BACKGROUND

Visualization of data is an ever-growing and developing field. Visualization of medical data is one important part of that developing field, as proficient visualization can benefit many different aspects of medicine including diagnosis, treatment, and more. Indeed, visualization of medical data has become richer and more varied over time due to the availability of electronic medical records (EMR). With the availability of EMR, for example, more patient information and larger patient cohorts are possible, resulting in much richer data analysis and visualization.

One important type of medical data is tumor heterogeneity, which can be described as differences between clusters or subclones of cancer cells within a single tumor. Although there are several types of differences between cancer cells, some of the most important differences are genetic mutations, which can lead to significant variations in the functioning of the cancer cells. Some cells within a single tumor may have a first set of genetic mutations that give rise to certain characteristics including therapy sensitive or resistance, and other cells within the same tumor may have another set of genetic mutations that give rise to different characteristics. Tumor heterogeneity can be an important factor in how a tumor is identified and treated, as well as how that tumor may respond to therapy.

Understanding and visualizing tumor heterogeneity remains a complicated challenge. While tumor heterogeneity can be quantified, displaying that quantification in a manipulatable and easy-to-understand format is more difficult. Current methods for visualizing tumor heterogeneity are not efficient or easy to understand. Additionally, current methods for visualizing tumor heterogeneity fail to incorporate possible therapies, especially in view of the quantified tumor heterogeneity.

Another important type of medical data is mortality data. For example, it is common to utilize mortality or survival of a patient cohort, given a common diagnosis of that patient cohort, over time. This provides an individual receiving that diagnosis with information about their mortality or survival odds for a similar time period. One common plot of survival is the Kaplan-Meier plot, which in the medical field graphs a calculation of the percentage of patients living for a certain amount of time after diagnosis or treatment. However, the Kaplan-Meier plot and visualizations like it are limited. They show data in only one form rather than encompassing the full array of information available from EMR.

### SUMMARY OF THE DISCLOSURE

Accordingly, there is a continued need for methods and systems for improved medical data visualization. Various embodiments and implementations herein are directed to a tumor visualization system that generates and provides a visual representation of both a patient's heterogenous tumor and a therapy effectiveness for that heterogenous tumor. The tumor visualization system receives information about a patient, including demographic and clinical information about the patient, an identification of the patient's tumor type, and a heterogeneity assessment of the patient's tumor. The heterogeneity assessment includes an analysis of heterogeneity of the patient's tumor and an identification of two or more subclones of the tumor, where the two or more subclones are differentiated by one or more genetic variations. The tumor visualization system obtains therapy effectiveness information for each of the two or more subclones of the tumor. The tumor visualization system then generates a visual representation of the heterogeneity assessment of the patient's tumor and the obtained therapy effectiveness information for each of the two or more subclones of the tumor, and provides that generated visual representation to a user via a user interface of the system.

Various embodiments and implementations herein are further directed to a survival statistics analysis system that generated and provides a visual representation of survival for a patient. The survival statistics analysis system receives information about a patient, including at least a patient diagnosis and a date of diagnosis. The system calculates, using the patient information and a reference survival dataset, one or more survival statistics for the patient for a first time period relative to a first historical cohort of patients. The system then generates a visual representation of the calculated one or more survival statistics and provides the generated visual representation via a user interface of the system. According to an embodiment, the generated visual representation of survival for a patient comprises one or more of a survival function graph augmented with mortality data, a median survival graph, a mortality risk graph, and a survival probability graph.

Generally, in one aspect, a method for visualizing heterogeneity of a patient's tumor and a therapy effectiveness for the patient's heterogenous tumor is provided. The method includes: (i) receiving information about a patient, the received information comprising demographic and clinical information about the patient, an identification of the patient's tumor type, and a heterogeneity assessment of the patient's tumor, the heterogeneity assessment comprising an analysis of heterogeneity of the patient's tumor and an identification of two or more subclones of the tumor; (ii) obtaining, based on the identification of the patient's tumor type and the heterogeneity assessment of the patient's tumor, therapy effectiveness information for each of the two or more subclones of the tumor; (iii) generating a visual representation of the heterogeneity assessment of the patient's tumor and the obtained therapy effectiveness information for each of the two or more subclones of the tumor; and (iv) providing, via a user interface of the system, the generated visual representation, wherein the visual representation comprises: 1. a tumor heterogeneity graph, comprising: (i) an identification of each of the two or more subclones of the patient's tumor, wherein the identification comprises an indication of a relative amount of each subclone in the patient's tumor; and (ii) a heterogeneity score indicating an overall complexity of tumor composition of the patient, wherein the heterogeneity score is visualized in an ordered data plot of heterogeneity scores of a same tumor type from a patient cohort, indicating a degree of tumor heterogeneity of the patient as a percentile rank within the cohort; and 2. a therapy effectiveness graph, comprising an indication of effectiveness of each of a plurality of different therapies for each of the two or more subclones of the patient's tumor.

According to an embodiment, the generated visual representation further comprises some or all of the demographic and clinical information about the patient.

According to an embodiment, the tumor heterogeneity graph further comprises an indication of a metastatic potential of each subclone in the patient's tumor.

According to an embodiment, the two or more subclones are differentiated by one or more molecular or histological variations. According to an embodiment, the tumor heterogeneity graph further comprises an identification of some or all of the one or more molecular or histological variations specific to each of the two or more subclones. According to an embodiment, the one or more molecular variations comprise one or more of genetic, epigenetic, transcriptomic, and proteomic variations, among other possible variations, including but not limited to single-nucleotide variants, indels, gene fusions, structural variants, gene/protein expression and methylation, among others.

According to an embodiment, each of the two or more subclones of the patient's tumor on the tumor heterogeneity graph are selectable, and wherein selecting a subclone results in a visual display of some or all of the one or more genetic variations specific to the selected subclone.

According to an embodiment, the therapy effectiveness graph further comprises an indication of effectiveness of a combination of two or more therapies for the two or more subclones of the patient's tumor.

According to an embodiment, the tumor heterogeneity graph and/or the therapy effectiveness graph further comprises one or more of side effect information for each of the plurality of different therapies and a cost of each of the plurality of different therapies.

According to another aspect is a system for providing a visualization of heterogeneity of a patient's tumor and a therapy effectiveness for the patient's heterogenous tumor. The system includes: patient information comprising at least demographic and clinical information about the patient, an identification of the patient's tumor type, and a heterogeneity assessment of the patient's tumor, the heterogeneity assessment comprising an analysis of heterogeneity of the patient's tumor and an identification of two or more subclones of the tumor; a therapy database; a processor configured to: (i) obtain, based on the identification of the patient's tumor type and the heterogeneity assessment of the patient's tumor, therapy effectiveness information for each of the two or more subclones of the tumor from the therapy database; and (ii) generate a visual representation of the heterogeneity assessment of the patient's tumor and the obtained therapy effectiveness information for each of the two or more subclones of the tumor; and a user interface configured to provide the generated visual representation, wherein the generated visual representation comprises: 1. a tumor heterogeneity graph, comprising: (i) an identification of each of the two or more subclones of the patient's tumor, wherein the identification comprises an indication of a relative amount of each subclone in the patient's tumor; and (ii) a heterogeneity score indicating an overall complexity of tumor composition of the patient, wherein the heterogeneity score is visualized in an ordered data plot of heterogeneity scores of a same tumor type from a patient cohort, indicating a degree of tumor heterogeneity of the patient as a percentile rank within the cohort; and 2. a therapy effectiveness graph, comprising an indication of effectiveness of each of a plurality of different therapies for each of the two or more subclones of the patient's tumor.

Generally, in another aspect, a method for providing a visual representation of survival for a patient is provided. The method includes: (i) receiving information about the patient, comprising at least a patient diagnosis; (ii) calculating, using the patient information and a reference survival dataset, one or more survival statistics for the patient for a first time period relative to a first historical cohort of patients; (iii) generating a visual representation of the calculated one or more survival statistics; and (iv) providing, via a user interface of the system, the generated visual representation. According to an embodiment, the generated visual representation comprises one or more of:
1. a survival function graph, the survival function graph comprising a single graph with both: (i) a plot of a Kaplan-Meier estimate for the first historical cohort for the first time period; and (ii) a plot of trailing mortality, comprising a percentage of mortality of the first historical cohort for a trailing time period, the trailing time period being a windowed segment of the first time period;
2. a median survival graph, the median survival graph comprising a single graph with (i) an identification of a median survival time for the first historical cohort for the first time period; and (ii) a plot of a percentage of mortality of the first historical cohort for a pre-median survival time period; and (iii) a plot of a percentage of survival of the first historical cohort for a post-median survival time period;
3. a mortality risk graph, the mortality risk graph comprising a plot of trailing (also called moving-window) mortality, comprising a percentage of mortality, for a trailing time period, of the first historical cohort relative to a number of survivors of the first historical cohort at a beginning of the trailing time period, the trailing time period being a windowed segment of the first time period; and
4. a survival probability graph, the survival probability graph comprising a plot of survival probability over the first time period, wherein the survival probability is calculated for each of a plurality of time points over the first time period by dividing a total number of survivors of the first historical cohort at the end of the first time period by a total number of survivors of the first historical cohort at the time point being calculated.

According to an embodiment, the first time period is 5 years.

According to an embodiment, the trailing time period is 6 months.

According to an embodiment, the survival function graph further comprises a final survivor probability estimate for the first time period.

According to an embodiment, the median survival graph further comprises a final survivor probability estimate for the first time period.

According to an embodiment, providing the generated visual representation comprises providing two or more of: (i) the survival function graph; (ii) the median survival graph; (iii) the mortality risk graph; and (iv) the survival probability graph.

According to an embodiment, the user interface is configured to allow a user to navigate between the two or more graphs.

According to an embodiment, the user interface is configured to display two or more of the graphs at once.

According to an embodiment, each graph is calculated using each of a plurality of different historical cohorts of patients, each of the plurality of different historical cohorts of patients comprising a different relevant condition, such as tumor heterogeneity index, mutational burden, BMI, etc., for the patients in that respective historical cohort.

According to another aspect is a system for providing a visual representation of survival for a patient. The system includes: patient information comprising at least a patient diagnosis; a reference survival dataset; a processor configured to: (i) receiving information about the patient, comprising at least a patient diagnosis and a date of diagnosis; (ii) calculate, using the patient information and a reference survival dataset, one or more survival statistics for the patient for a first time period relative to a first historical cohort of patients; and (iii) generate a visual representation of the calculated one or more survival statistics; and a user interface configured to provide the generated visual representation. According to an embodiment, the generated visual representation comprises one or more of:
1. a survival function graph, the survival function graph comprising a single graph with both: (i) a plot of a Kaplan-Meier estimate for the first historical cohort for the first time period; and (ii) a plot of trailing mortality, comprising a percentage of mortality of the first historical cohort for a trailing time period, the trailing time period being a windowed segment of the first time period;
2. a median survival graph, the median survival graph comprising a single graph with (i) an identification of a median survival time for the first historical cohort for the first time period; and (ii) a plot of a percentage of mortality of the first historical cohort for a pre-median survival time period; and (iii) a plot of a percentage of survival of the first historical cohort for a post-median survival time period;
3. a mortality risk graph, the mortality risk graph comprising a plot of trailing mortality, comprising a percentage of mortality, for a trailing time period, of the first historical cohort relative to a number of survivors of the first historical cohort at a beginning of the trailing time period, the trailing time period being a windowed segment of the first time period; and
4. a survival probability graph, the survival probability graph comprising a plot of survival probability over the first time period, wherein the survival probability is calculated for each of a plurality of time points over the first time period by dividing a total number of survivors of the first historical cohort at the end of the first time period by a total number of survivors of the first historical cohort at the time point being calculated.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for generating and providing a visual representation of tumor heterogeneity and/or therapy effectiveness, in accordance with an embodiment.

FIG. 2 is a schematic representation of a tumor visualization system, in accordance with an embodiment.

FIG. 3 is an example of a visual representation including a patient information panel and a tumor heterogeneity graph panel, in accordance with an embodiment.

FIG. 4 is an example of a visual representation of a tumor heterogeneity graph, in accordance with an embodiment.

FIG. 5 is an example of a tumor heterogeneity graph panel, in accordance with an embodiment.

FIG. 6 is an example of a tumor heterogeneity graph panel, in accordance with an embodiment.

FIG. 7A is an example of a visual representation including a therapy effect panel, in accordance with an embodiment.

FIG. 7B is an example of a visual representation of a drug selection panel, in accordance with an embodiment.

FIG. 8 is an example of a therapy effectiveness panel, in accordance with an embodiment.

FIG. 9 is an example of a therapy effectiveness graph for a plurality of subclones, in accordance with an embodiment.

FIG. 10 is an example of a therapy effectiveness graph for a plurality of subclones, in accordance with an embodiment.

FIG. 11 is an example of a therapy effectiveness graph for a plurality of subclones, in accordance with an embodiment.

FIG. 12 is an example of a drug selection panel, in accordance with an embodiment.

FIG. 13 is an example of a therapy effectiveness graph for a plurality of subclones, in accordance with an embodiment.

FIG. 14 is an example of a therapy effectiveness panel, in accordance with an embodiment.

FIG. 15 is an example of a drug selection panel, in accordance with an embodiment.

FIG. 16 is an example of a drug selection panel, in accordance with an embodiment.

FIG. 17 is an example of a therapy effectiveness graph for a plurality of subclones, in accordance with an embodiment.

FIG. 18 is an example of a therapy effectiveness graph for a plurality of subclones, in accordance with an embodiment.

FIG. 19 is an example of a therapy effectiveness graph for a plurality of subclones, in accordance with an embodiment.

FIG. 20 is an example of a therapy effectiveness panel, in accordance with an embodiment.

FIG. 21 is an example of a portion of a therapy effectiveness panel, in accordance with an embodiment.

FIG. 22 is a flowchart of a method for generating and providing a visual representation of survival for a patient, in accordance with an embodiment.

FIG. 23 is a schematic representation of a survival statistics analysis system, in accordance with an embodiment.

FIG. 24A is an example of a survival function graph, in accordance with an embodiment.

FIG. 24B is an example of a survival function graph, in accordance with an embodiment.

FIG. 25A is an example of a median survival graph, in accordance with an embodiment.

FIG. 25B is an example of a median survival graph, in accordance with an embodiment.

FIG. 26A is an example of a mortality risk graph, in accordance with an embodiment.

FIG. 26B is an example of a mortality risk graph, in accordance with an embodiment.

FIG. 27A is an example of a survival probability graph, in accordance with an embodiment.

FIG. 27B is an example of a survival probability graph, in accordance with an embodiment.

FIG. 28 is an example of a survival function graph, in accordance with an embodiment.

FIG. 29 is an example of a median survival graph, in accordance with an embodiment.

FIG. 30 is an example of a mortality risk graph, in accordance with an embodiment.

FIG. 31 is an example of a survival probability graph, in accordance with an embodiment.

### DETAILED DESCRIPTION OF EMBODIMENTS

The present disclosure describes various embodiments of a systems and methods for improved medical data visualization.

According to one aspect is a system and method configured to generate and provide a visual representation of a patient's tumor. More generally, Applicant has recognized and appreciated that it would be beneficial to provide improved methods and systems for the visualization of tumor heterogeneity. Accordingly, a tumor heterogeneity system is described which generates and provides a visual representation of tumor heterogeneity. The tumor heterogeneity system receives information about a patient, including demographic and clinical information about the patient, an identification of the patient's tumor type, and a heterogeneity assessment of the patient's tumor. The heterogeneity assessment includes an analysis of heterogeneity of the patient's tumor and an identification of two or more subclones of the tumor, where the two or more subclones are differentiated by one or more molecular and/or histological variations. The tumor visualization system obtains therapy effectiveness information for each of the two or more subclones of the tumor. The tumor visualization system then generates a visual representation of the heterogeneity assessment of the patient's tumor and the obtained therapy effectiveness information for each of the two or more subclones of the tumor, and provides that generated visual representation to a user via a user interface of the system. According to an embodiment, the generated visual representation of tumor heterogeneity comprises a tumor heterogeneity graph and a therapy effectiveness graph.

It should be understood that while the systems and methods described or otherwise envisioned herein focus on the utilization of the system for visualizing tumor heterogeneity solely for purposes of explanation, the visualization systems and methods are applicable to anything for which heterogeneity could be quantified and displayed. This can include medical systems as well as non-medical systems.

According to another aspect is a system and method configured to generate and provide a visual representation of survival. More generally, Applicant has recognized and appreciated that it would be beneficial to provide improved methods and systems for the visualization of patient survival probability over time. Accordingly, a survival statistics analysis system is described which generates and provides a visual representation of survival for a patient. The survival statistics analysis system receives information about a patient, including at least a patient diagnosis and a date of diagnosis. The system calculates, using the patient information and a reference survival dataset, one or more survival statistics for the patient for a first time period relative to a first historical cohort of patients. The system then generates a visual representation of the calculated one or more survival statistics and provides the generated visual representation via a user interface of the system. According to an embodiment, the generated visual representation of survival for a patient comprises one or more of a survival function graph, a median survival graph, a mortality risk graph, and a survival probability graph.

It should be understood that while the systems and methods described or otherwise envisioned herein focus on the utilization of the system for survival statistics, solely for purposes of explanation. However, while survival data is used to illustrate the idea, the systems and methods applicable to any time-to-event data, where events may include, e.g., injury, onset of illness, recovery from illness, recurrence of a disease, discharge from hospital or failure of device or termination of relationship or attention, and a wide variety of other data. Typically, time-to-event data focus on the time elapsing before an event is experienced, often known as survival data in statistics. Notably, time-to-event data may be based on events other than death, such as recurrence of a disease event or discharge from hospital, among other events.

According to an embodiment, the systems and methods described or otherwise envisioned herein can, in some non-limiting embodiments, be implemented as an element for a commercial product for medical data such as Philips® IntelliSpace (available from Koninklijke Philips NV, the Netherlands), or as an element for a commercial product for patient analysis or monitoring, or any suitable system.

The methods and systems described or otherwise envisioned herein provide numerous advantages over existing data generation and visualization methods and systems. A medical display that can provide this novel format of patient data/information, including the tumor heterogeneity graph and a therapy effectiveness graph, and/or the claimed novel graphs of survival function, median survival, mortality risk, and/or survival probability graph, provides inventive solutions to the problem of overwhelming information and overly cluttered visualizations and computer displays. For example, a 2020 study by Philips (“Future Health Index 2020”) showed that 35% of younger healthcare professionals don't know how to use digital patient data to inform patient care, and that 35% of younger healthcare professionals are overwhelmed by the amount of digital patient data, including the amount of information shared via patient monitors and visualization systems. This is not an unusual result; many other surveys have found that physicians and clinicians are overwhelmed with information, and that patient care suffers as a result. The claimed data generation and visualization methods and systems provide simplified mechanisms for displaying massive amounts of data, including tumor homogeneity data (which can come from thousands of data points), therapy effectiveness, and survival statistics. Rather than a generic computer system providing generic information, as claimed the data generation and visualization methods and systems described or otherwise envisioned herein provide very specific inventive solutions to the ongoing problem of data visualization. The claimed data generation and visualization methods and systems are similarly not simply a computerized version of paper records. Rather, the visualizations are novel computer-based visualizations that enable data visualization and manipulation that cannot be done with paper records.

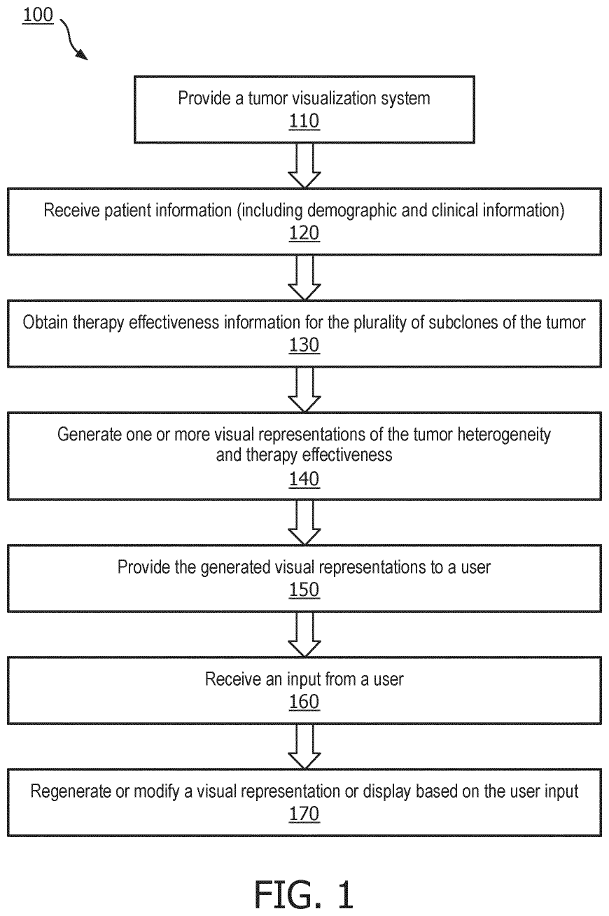

Referring to FIG. 1, in one embodiment is a flowchart of a method 100 for generating and providing a visual representation of tumor heterogeneity using a tumor visualization system. The methods described in connection with the figures are provided as examples only, and shall be understood to not limit the scope of the disclosure. The tumor visualization system can be any of the systems described or otherwise envisioned herein. The tumor visualization system can be a single system or multiple different systems.

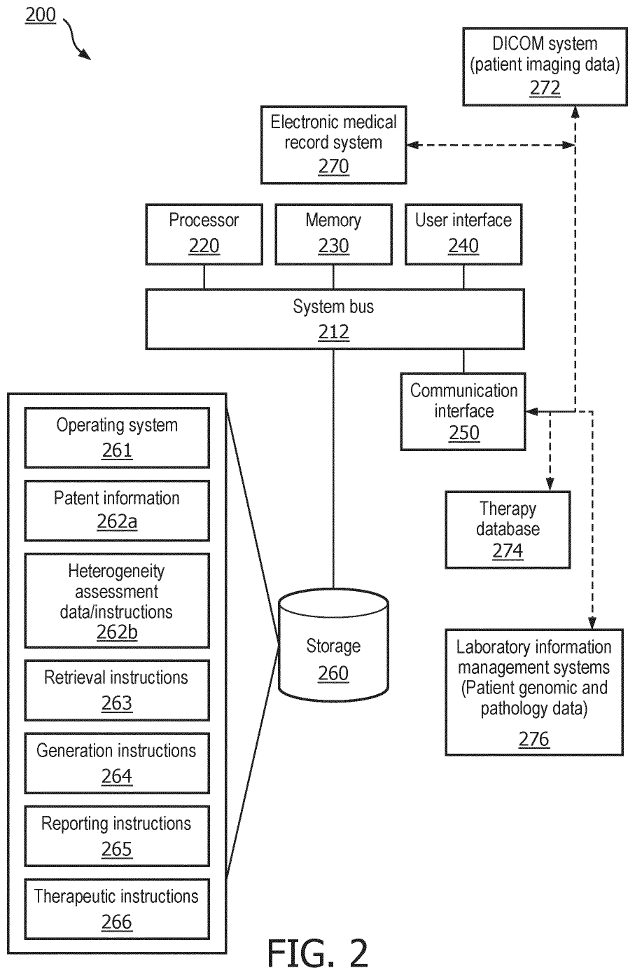

At step 110 of the method, a tumor visualization system 200 is provided. Referring to an embodiment of a tumor visualization system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, tumor visualization system 200 can be any of the systems described or otherwise envisioned herein. Other elements and components of tumor visualization system 200 are disclosed and/or envisioned elsewhere herein.

At step 120 of the method, the tumor visualization system receives information about a patient. The patient information can be any information about the patient that the tumor visualization system can or may utilize for analysis as described or otherwise envisioned herein. According to an embodiment, the patient information comprises one or more of demographic and clinical information about the patient, an identification of the patient's tumor type, and a heterogeneity assessment of the patient's tumor. Other information is possible.

According to an embodiment, the demographic and clinical information about the patient may comprise anything related to the patient, including but not limited to information about the patient such as name, age, address, body mass index (BMI), and any other demographic information. The clinical information may also comprise diagnosis information for the patient, which may be any information about a medical diagnosis for the patient, historical and/or current. The clinical information may also comprise a medical history of the patient which may be any historical admittance or discharge information, historical treatment information, historical diagnosis information, historical exam or imaging information, and/or any other information.

According to an embodiment, the identification of the patient's tumor type comprises an identification of the patient's cancer type. For example, the cancer type can be any known type of cancer, including but not limited to breast cancer, lung cancer, melanoma, endometrial cancer, kidney cancer, colorectal cancer, leukemia, non-Hodgkin lymphoma, pancreatic cancer, prostate cancer, thyroid cancer, and many more. In addition to a high-level cancer type, the identification of the patient's tumor type can comprise a more specific identification, such as head-neck squamous cell carcinoma (HNSC), non-small cell lung cancer (NSCLC), renal cell carcinoma (RCC), familial colorectal cancer (FCC), and many more.

According to an embodiment, in addition to the high-level cancer type and/or the more specific identification of the cancer type, the identification of the patient's tumor type can comprise information about the severity or other qualitative or quantitative assessment of the patient's cancer. For example, the information may comprise a number staging of the patient's tumor (stage 0, stage 1, stage 2, stage 3, and stage 4 where each stage is known in the art to be associated with specific characteristics). The information may comprise the TNM (tumor (T), node (N), and metastasis (M)) staging system in which the T (numbers 1 through 4) describes the size of the tumor, the N (numbers 0 to 3) describe the involvement of lymph nodes, and the M (numbers 0 or 1) describes whether the tumor has metastasized or not. The information may comprise a grade (grade 1, grade 2, and grade 3) based on what the cells look like under a microscope, where a lower grade indicates a slower-growing cancer, and a higher grade indicates a faster-growing cancer. Other qualitative or quantitative assessments of the patient's cancer are possible.

According to an embodiment, the identification of the patient's tumor type can be extracted or deduced from medical records, the received patient demographic and clinical information, and/or from other sources of information about the patient.

According to an embodiment, the heterogeneity assessment of the patient's tumor comprises a qualitative or quantitative assessments of the heterogeneity of the patient's tumor. A single tumor can be heterogenous in several different ways. For example, genetic, epigenetic, microenvironmental, transcriptomic, proteomic, histological, and imaging heterogeneities are known to exist—and co-exist—in tumors and can be linked with linked with phenotypic diversity (including behavioral phenotypes as well as responsiveness to therapy). Notably, tumor heterogeneity can be detected within the same tumor (so-called intra-tumor heterogeneity), as well as between a primary tumor and metastatic lesions. For purposes of this disclosure, the term heterogeneity refers to any difference within the same tumor or between a primary tumor and metastatic lesions, among other possibilities. Tumor heterogeneity has been detected in nearly every type of cancer and tumor, and can change and evolve as the cancer develops over time.

There are several methods for analyzing the heterogeneity of a tumor. One method for analyzing the heterogeneity of a tumor is through imaging of the tumor. Different imaging modalities including but not limited to X-ray, ultrasound (US), computed tomography (CT), magnetic resonance imaging (MRI), and positron emission tomography (PET), can be used singly or in combination to image the tumor, depending on the tumor type and physical location in or on the patient. The imaging can then be utilized for a qualitative and/or quantitative assessment of the tumor's heterogeneity.

According to another embodiment, the heterogeneity of the tumor may be analyzed with genetic investigative methods and systems, where “genetic” can refer to at least genetic, epigenetic, and transcriptomic analysis or variation. Among other investigative methods, the patient's tumor can be examined with molecular testing to determine a qualitative and/or quantitative assessment of the tumor's heterogeneity. That molecular testing may comprise sequencing or other genetic, epigenetic, or transcriptomic analysis of one or more cells from one or more regions of the tumor.

For example, single-cell molecular testing approaches will examine individual cells from one or more regions of the tumor (or from the primary site and one or more metastases). Multi-region molecular testing approaches will examine one or more cells from one or more regions of the tumor (or from the primary site and one or more metastases). Other methods of sampling are possible, including representative sampling (Rep-Seq) and liquid biopsy, among others. Heterogeneity can also be assessed by applying specific computational analysis algorithms on bulk sequencing data.

According to an embodiment, the genetic, epigenetic, microenvironmental, transcriptomic, and/or proteomic analysis of tumor heterogeneity results in an identification of at least two subclones of the tumor. A subclone can be defined as a variation-either within a single tumor (intra-tumor heterogeneity) or between a primary tumor and a metastasized tumor-identified by a heterogeneity analysis. For example, a heterogeneous tumor thus comprises at least two different subclones, although there many be more than two subclones. The threshold for labeling a variation as a subclone may depend on a user, programming of the system, standards within the art, and other parameters.

According to an embodiment, the at least two different subclones of the heterogeneous tumor can be defined in a variety of different ways, including based on one or more of a genetic, epigenetic, microenvironmental, transcriptomic, proteomic, histological, and/or imaging analysis. For example, the at least two different subclones of the heterogeneous tumor can be defined by epigenetic differences such as different histone modifications, microenvironmental differences, transcriptomic differences, and/or proteomic differences, among other differences. In accordance with one embodiment, the at least two different subclones of the heterogeneous tumor are defined by genetic variations, including but not limited to genetic mutations, among other possible genetic variations. Accordingly, the two or more subclones can be differentiated by one or more genetic variations.

According to an embodiment, the patient information is received from one or a plurality of different sources. According to an embodiment, the patient information is received from, retrieved from, or otherwise obtained from an electronic medical record (EMR) database or system 270. The EMR database or system may be local or remote. The EMR database or system may be a component of the tumor visualization system, or may be in local and/or remote communication with the tumor visualization system. The received patient information may be utilized immediately, or may be stored in local or remote storage for use in further steps of the method.

At step 130 of the method, the tumor visualization system 200 receives, retrieves, or otherwise obtains therapy effectiveness information for each of the two or more subclones of the tumor. This therapy effectiveness information is identified and obtained based on the received identification of the patient's tumor type and the received heterogeneity assessment of the patient's tumor.

Therapy effectiveness can be based on known effectiveness of one or more therapies for a tumor type, as well as for an identified subclone type. For example, there may be a database of possible cancer therapies associated with a known or estimated effectiveness of some or all of these possible cancer therapies in treating an identified subclone type of a specific tumor type (where “treating” can be a variety of outcomes including cell death, inhibition of growth, and other outcomes). For example, where a subclone is defined by a genetic mutation X, it may be known in the art or in the therapy database (now or in the future) how subclones with genetic mutation X respond to a variety of different therapies.

According to an embodiment, the tumor visualization system can retrieve information from a therapy database based on the received heterogeneity assessment of the patient's tumor, such as a result from the qualitative and/or quantitative genetic, epigenetic, microenvironmental, transcriptomic, proteomic, histological, and/or imaging analysis assessment of the tumor. As just one example, the tumor visualization system can retrieve information from a therapy database by searching for one or more genetic mutations associated with a subclone, in order to retrieve therapies associated with those one or more genetic mutations. Notably, a therapy can be associated with a genetic mutation (or other identification of subclone heterogeneity) based on the subclone responding favorably to that therapy, or based on the subclone responding unfavorably to that therapy. These can both be important information for the tumor visualization system as described or otherwise envisioned herein.

According to an embodiment, the therapy effectiveness information is received from one or a plurality of different sources. According to an embodiment, the therapy effectiveness information is received from, retrieved from, or otherwise obtained from a therapy database or system 274. The therapy database or system may be local or remote. The therapy database or system may be a component of the tumor visualization system, or may be in local and/or remote communication with the tumor visualization system. The received therapy effectiveness information may be utilized immediately, or may be stored in local or remote storage for use in further steps of the method.

At step 140 of the method, the tumor visualization system utilizes the received heterogeneity assessment of the patient's tumor and the obtained therapy effectiveness information for each of the two or more subclones of the tumor to generate a visual representation. The visual representation comprises a representation of the heterogeneity assessment of the patient's tumor, and a representation of the obtained therapy effectiveness information. The visual representation may be generated immediately after the heterogeneity assessment and therapy effectiveness information are received, or may be performed using stored information. The system may generate the one or more visual representations on demand or as a result of programming or other automation. According to an embodiment, the system generates the one or more visual representations in response to a user input. For example, a physician or other healthcare professional may request the information. Alternatively, the system may be partially or entirely automated, such that the one or more visual representations are automatically calculated. For example, the system may be a standalone system or a component of a medical data or analysis system, which may comprise a user interface that displays information about a patient. This information may comprise one or more of the visual representations described or otherwise envisioned herein.

The one or more visual representations of the heterogeneity assessment and therapy effectiveness information are generated pursuant to any of the methods described or otherwise envisioned herein. For example, described below are several different non-limiting methods and examples of visual representations of heterogeneity assessment and therapy effectiveness information, and thus the system may be designed or programmed to generate such visual representations using known methods and mechanisms for graphing data. These and many other methods may be utilized by the system.

Once generated, the one or more visual representations of the heterogeneity assessment and therapy effectiveness information may be utilized immediately, or may be stored in local or remote storage for use in further steps of the method.

At step 150 of the method, the tumor visualization system provides, via a user interface, the generated one or more visual representations of the heterogeneity assessment and therapy effectiveness information. For example, a visual representation may be displayed to a medical professional or other user, including the patient, via the user interface of the system. The generated one or more visual representations may be provided to a user via any mechanism for display, visualization, or otherwise providing information via a user interface. According to an embodiment, the information may be communicated by wired and/or wireless communication to a user interface and/or to another device. For example, the system may communicate the information to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands. As just one non-limiting example, the user interface may be a component of a patient monitoring system.

According to an embodiment, the display may further comprise patient information such as demographic and clinical information about the patient, a diagnosis for the patient, medical history of the patient, and/or any other information.

According to an embodiment, as described herein, some or all of the displayed information may be manipulatable, in response to user input provided via the user interface.

The tumor visualization system provides, via a user interface, a generated visual representation of a heterogeneity assessment of the patient's tumor as well as therapy effectiveness information for the heterogeneous tumor, which can be any of a variety of different statistics and visual representations. According to one embodiment, the visual representation comprises a tumor heterogeneity graph. The tumor heterogeneity graph can comprise, for example, an identification or other indication of each of the two or more subclones of the patient's tumor. According to an embodiment, that identification comprises an indication of a relative amount of each subclone in the patient's tumor, with respect to each other and/or to with respect to the entire tumor. The tumor heterogeneity graph can further comprise, for example, a tumor heterogeneity “score” or “index” (referred herein as a “tumor heterogeneity score”) for the patient's tumor. There are many different ways to evaluate or calculate a tumor heterogeneity score for a tumor. According to one embodiment, the tumor heterogeneity score is calculated based on the methodology in which the heterogeneity is determined. For example, the score may be based on parameters derived from an imaging modality, based on genetic variations such as mutational burden or load, or based on any other method.

The tumor heterogeneity graph can further comprise, for example, an ordered data plot of heterogeneity scores for the same tumor type based on a cohort of other patients, which can be obtained by the system or provided in the patient information or heterogeneity assessment. The ordered data plot of tumor heterogeneity scores can include an identification on the plot of a tumor heterogeneity score for the patient, indicating the degree of tumor heterogeneity of the patient as a percentile rank within the cohort.

According to an embodiment, the tumor heterogeneity graph further comprises an identification of some or all of the one or more variations specific to each of the two or more subclones, such as for example the one or more molecular and/or histological variations specific to a subclone.

The tumor visualization system further provides, via the user interface, a generated therapy effectiveness graph which is a visual representation of the therapy effectiveness information for the heterogeneous tumor. The therapy effectiveness graph comprises, for example, an indication of effectiveness of each of a plurality of different therapies for each of the two or more subclones of the patient's tumor. Notably, effectiveness can mean a therapy with a favorable or positive outcome (such as effectively targeting or treating the cells of the cancer subclone including but not limited to killing, neutralizing, or stopping the cells of the cancer subclone), a therapy with a neutral outcome, or a therapy with an unfavorable or negative outcome (such as not targeting or treating the cells of the cancer subclone).

According to one embodiment, the therapy effectiveness graph further comprises an indication of effectiveness of a combination of two or more therapies for the two or more subclones of the patient's tumor. For example, the therapy effectiveness graph may comprise—automatically or in response to user input—a grouping of cancer therapies that can favorably target and/or treat most or all of the identified tumor subclones. According to an embodiment, the cancer visualization system can be programmed or designed to identify a therapy combination with a most favorable predicted outcome for most or all of the identified tumor subclones.

At step 160 of the method, a user provides input about the displayed one or more visualizations via the user interface of the tumor visualization system 200. The input can be any input, including but not limited to a command to modify a visualization, to add a visualization, to remove a visualization, and/or any other input or command. For example, the user interface may be configured to allow a user to navigate between two or more visualizations. The user interface may be configured to display one, two, or more visualizations at one time. The user may be a healthcare professional, the patient, and/or any other user.

According to another embodiment, at step 160 of the method, a user provides input to the system in response to the information provided via the reporting mechanisms. For example, the clinician can select a therapy to be administered to the patient, which can then be reported to a clinical decision support system or patient management system, optionally together with the evidence of all available options, for future auditing purposes among other things.

Thus, at step 170 of the method, the tumor visualization system regenerates one or more visualizations based on the received input, such as returning to a previous step of the method and generating the visualization based on the input. Alternatively, the system may modify a visualization based on the received input. As just one example, the user may request information about one or more subclones, or one or more therapies or therapy combinations. This might necessitate that the system recalculate and/or regenerate the visualizations, which can then be displayed via the user interface. Many other modifications are possible.

### EXAMPLES

Described below are examples of possible applications of the methods and systems described or otherwise envisioned herein. These examples are provided only as a possible embodiment of the methods and systems described or otherwise envisioned herein, and therefore do not limit or prohibit other possible variations and embodiments.

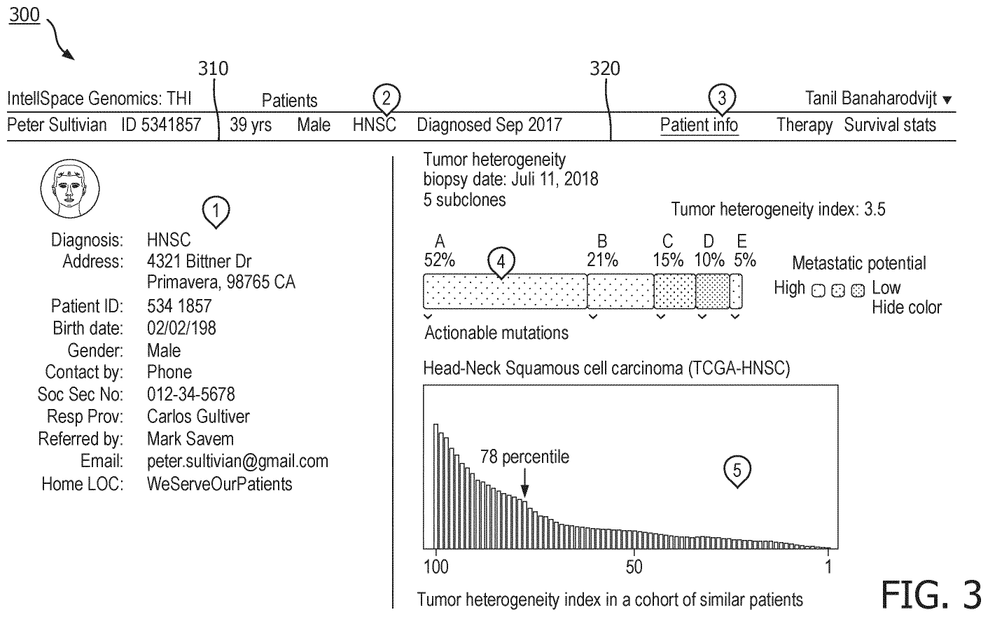

Referring to FIG. 3, in one embodiment, is a visual representation 300 generated by the tumor visualization system. The visual representation 300 comprises a patient information panel 310 and a tumor heterogeneity graph panel 320. According to this embodiment, the patient information panel 310 comprises some or all of the received patient information, including but not limited to an identification of the tumor type (HNSC), an address, patient identification information, date of birth, gender, social security number, primary physician, email address, and/or other information.

Referring to FIG. 5 is a larger representation of the tumor heterogeneity graph panel 320 of FIG. 3. The tumor heterogeneity graph panel comprises a graph of tumor heterogeneity 510. In this embodiment, the graph of tumor heterogeneity comprises a bar graph 510. The bar graph shows the five different subclones (subclone A, subclone B, subclone C, subclone D, and subclone E) and with their relative amount in the tumor (52%, 21%, 15%, 10%, and 2%, respectively).

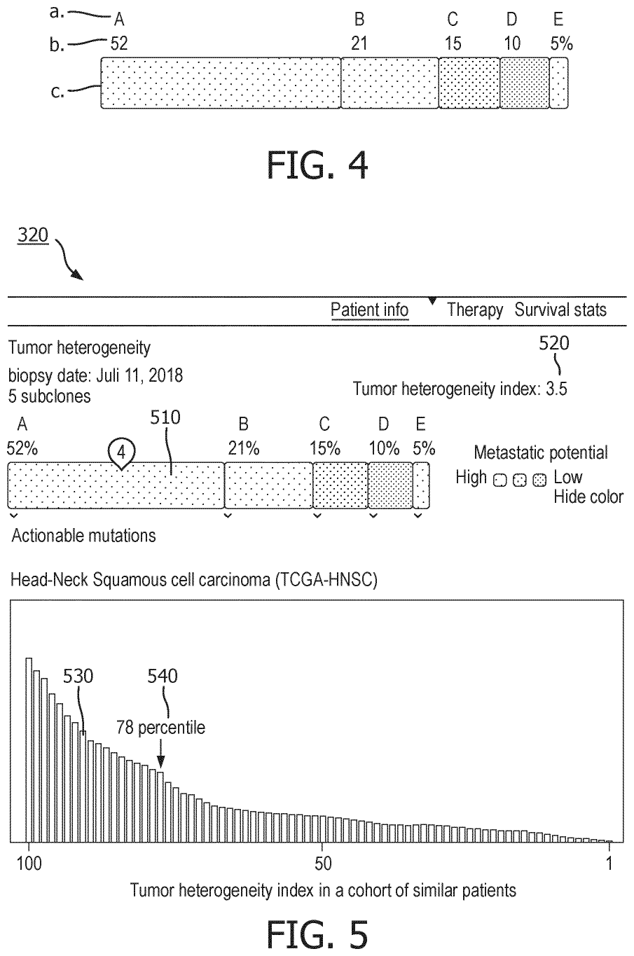

Referring to FIG. 4, in one embodiment, is a larger representation of the bar graph 510 of tumor heterogeneity showing the five different subclones (subclone A, subclone B, subclone C, subclone D, and subclone E). The Tumor Heterogeneity Bar (THB) provides basic information about relative sizes of the cancer subclones. Each block (c) represents a subclone and is marked with an identifying label (a) and optionally additional information such as specific size percentage (b).

Returning to FIG. 5, as shown in the graph, in accordance with an embodiment, some or all of the subclones may be selectable or otherwise able to be interacted with in order to reveal more information. For example, a subclone may be clicked, selected, or otherwise identified for information expansion. Clicking or selecting “Actionable mutations” in the graph would, for example, generate a list of one or more genetic mutations that can be affected by or can effect or affect therapy information or therapies. In other embodiments in which the subclones are identified by other methods such as histology and imaging, clicking or selecting a subclone may reveal other differentiators based on that method.

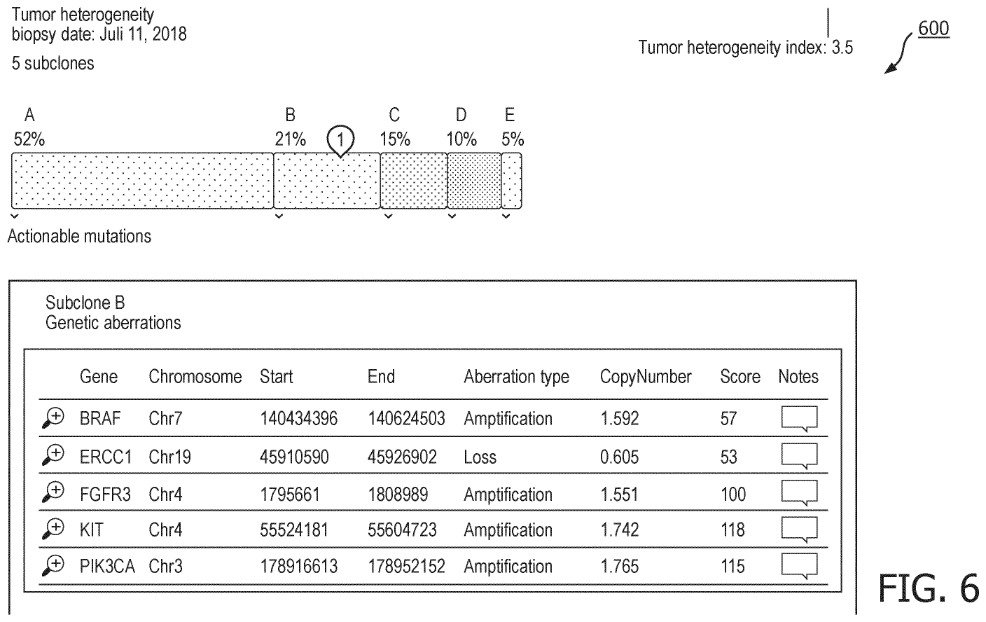

Referring to FIG. 6, in one embodiment, is a modification 600 of the tumor heterogeneity graph panel in which a user has clicked, selected, or otherwise identified subclone B for information expansion. As these subclones were identified using genetic analysis, the information expansion shows a list of one or more genetic mutations (in particular, five different mutations (i.e., “Genetic aberrations”)) that characterize this subclone. Other information in addition to the identification of the mutations can be provided, including but not limited to chromosome number, start and stop location, mutation type, copy number, score, notes, and other information.

Returning to the tumor heterogeneity graph panel 320 in FIG. 5, as shown in the graph and in accordance with an embodiment, some or all of the subclones may comprise information about metastatic potential, or other information. In this example, the graph of tumor heterogeneity 510 comprises color coding that indicates a metastatic potential (ranging from “High” to “Low”) for the five different subclones. For example, subclone D has a very low metastatic potential, while subclone A has a high metastatic potential. Other methods of conveying this information, such as patterning, sizing, or other methods, are possible. According to an embodiment, metastatic potential can be obtained by the system or provided in the patient information or heterogeneity assessment.

As shown in the graph, in accordance with an embodiment, the tumor heterogeneity graph panel can further comprise a tumor heterogeneity score 520 for the patient's tumor. As described above, there are many different ways to evaluate or calculate a tumor heterogeneity score for a tumor. According to one embodiment, the tumor heterogeneity score is calculated based on the methodology in which the heterogeneity is determined. Here, the determined tumor heterogeneity score for this patient's tumor is 3.5.

As shown in the graph, in accordance with an embodiment, the tumor heterogeneity graph panel can further comprise an ordered data plot 530 of tumor heterogeneity scores for the patient's tumor type, which can be obtained by the system or provided in the patient information or heterogeneity assessment. The ordered data plot of tumor heterogeneity scores can include an identification 540 on the plot of the tumor heterogeneity score for the patient's tumor. In this example, the tumor heterogeneity graph panel comprises an ordered data plot 430 of tumor heterogeneity scores for head-neck squamous cell carcinoma (HNSC), for a cohort of similar patients. In this example, the patient's tumor heterogeneity score of 3.5 falls within the 78th percentile of this cohort.

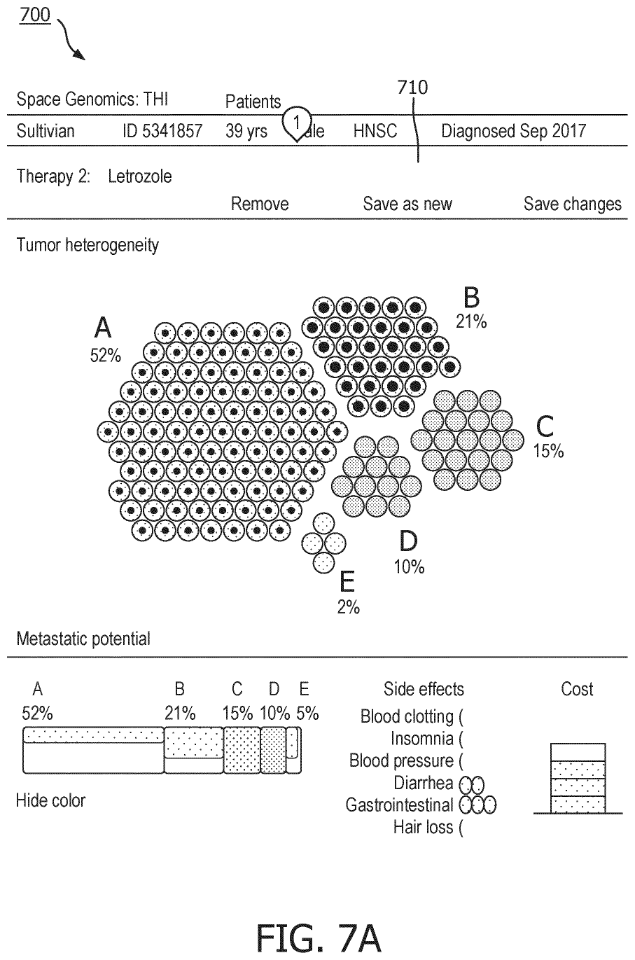

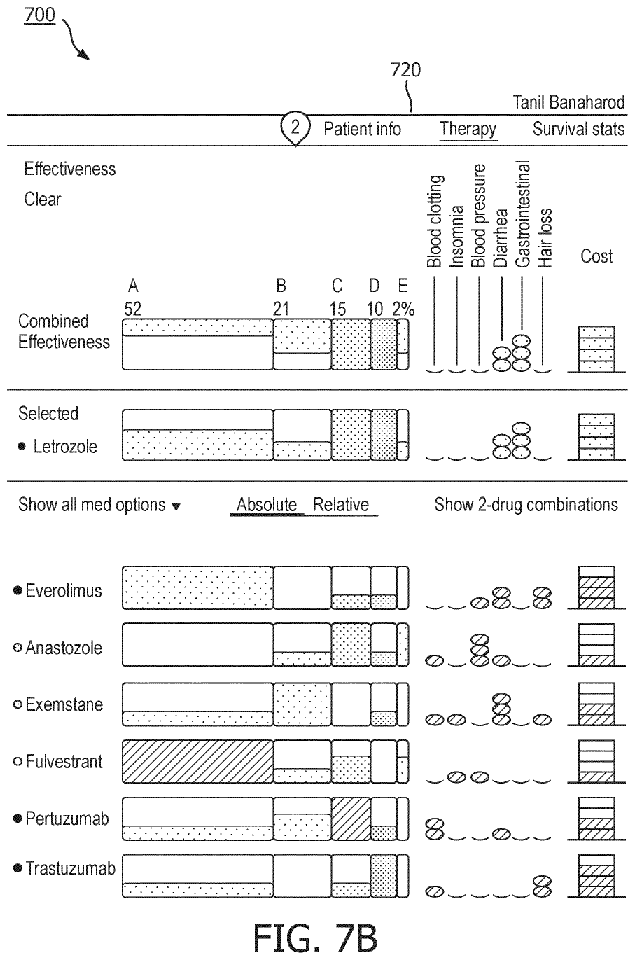

Referring to FIGS. 7A and 7B, in one embodiment, is a visual representation 700 generated by the tumor visualization system. The visual representation 700 comprises a therapy effectiveness panel 710 and a drug selection panel 720. According to this embodiment, the therapy effectiveness panel 710 comprises information about an effect of one or more therapies on one or more subclones, and shows the combined effectiveness of the one or more multiple drugs selected in the drug selection panel 720. According to this embodiment, the drug selection panel 720 comprises information about the effect of one or more therapies (here, one or more drug therapies) on one or more subclones.

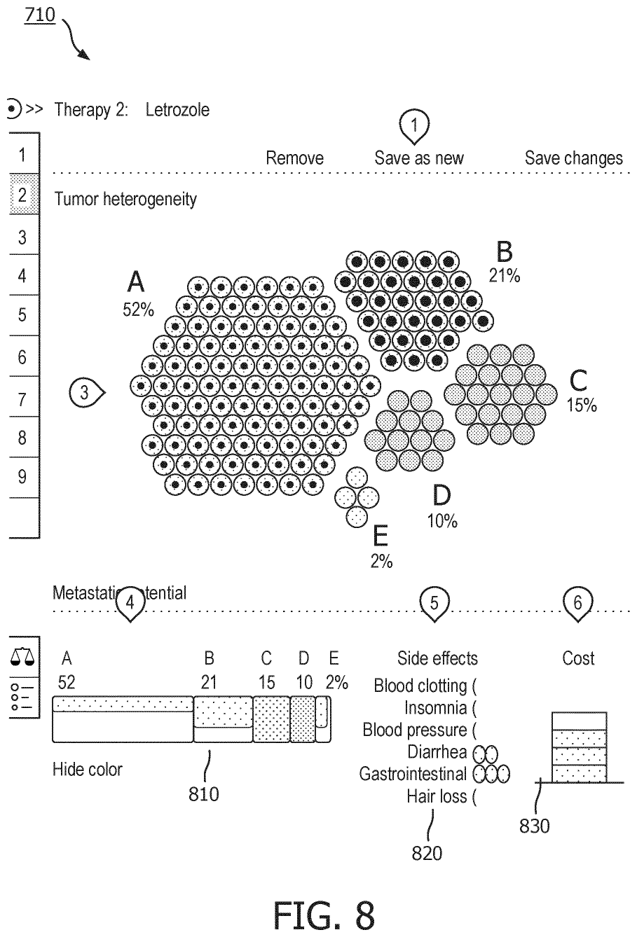

Referring to FIG. 8, in one embodiment, is a larger representation of the therapy effectiveness panel 710 of FIG. 7A. The therapy effectiveness panel 810 depicts the five different subclones (subclone A, subclone B, subclone C, subclone D, and subclone E) in this particular example, along with an indication of the effectiveness of a selected therapy (Letrozole) on each of the subclones. This effectiveness measure can be identified or displayed in a wide variety of ways, including a bar graph, line graph, or a variety of other graphs and display mechanisms. In the example shown in FIG. 8, the five different subclones are displayed as bubbles with a size of the subclone shown in relation to the totality of the tumor (52%, 21%, 15%, 10%, and 2%, respectively). The effectiveness measure is shown in this example via a filling (or lack thereof) of the bubbles of each subclone. For example, the selected therapy (Letrozole) is very effective against subclone D and the bubbles are not filled at all. For example, this can represent that the therapy will kill or otherwise strongly affect (e.g., “shrink”) subclone D. The therapy is not effective against subclone C (e.g., subclone C is resistant), and therefore the bubbles are completely filled in. The therapy works with varying success for subclones A, B, and E as shown by the bubble filling. Many other methods of displaying effectiveness are possible.

As shown in the graph, in accordance with an embodiment, the therapy effectiveness panel 710 can display other information. For example, the therapy effectiveness panel in this example comprises metastatic potential information 810 about the subclones, side effect information 820 about the selected therapy (Letrozole), and cost of the selected therapy 830. This and other information is possible. In one example, the metastatic potential is visualized by both the size of the shaded block at the top of each subclone indicating the proportion of cells resistant to the selected therapy and the shades of colors indicating the aggressiveness of the subclone.

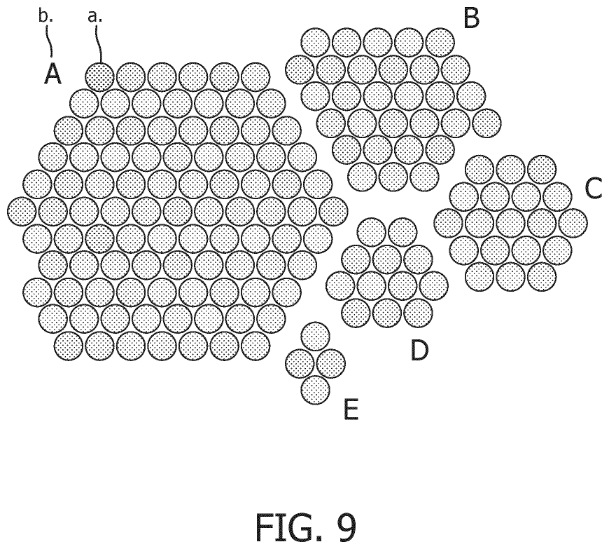

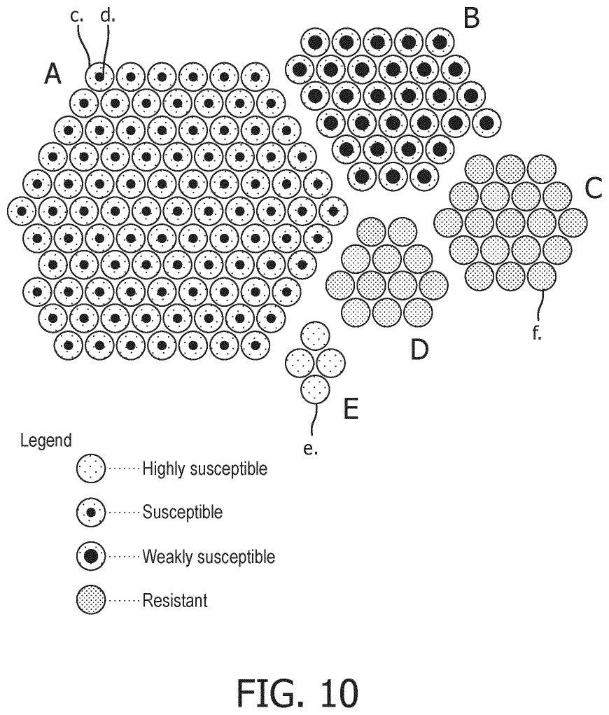

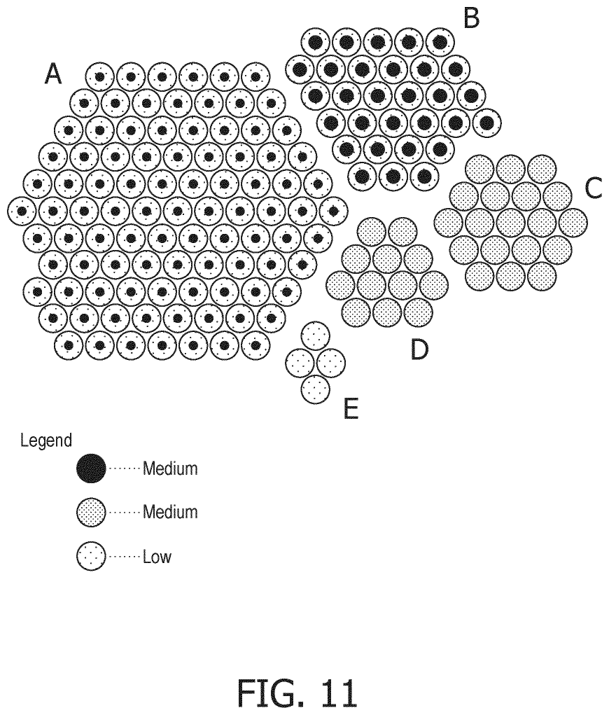

Referring to FIG. 9, in one embodiment, is an example of a possible visualization of tumor heterogeneity, using a hexagonal grid with cellular groupings (a) depicting relative sizes of the tumor subclones, and with labeling (b). This possible visualization creates framework for representing more specific information about medication susceptibility and metastatic potential of each subclone, as shown in FIGS. 10 and 11.

Referring to FIG. 10, in one embodiment, is an example of a possible visualization of tumor heterogeneity, using a hexagonal grid with cellular groupings for medication susceptibility. The spots at the center of the grid cells (d) provide visual clues about how each of the subclones may react to a medication. The degree of shrinkage of the spots in relation to the grid cells (c) size represents drug susceptibility of the subclone. In extreme cases the center spot may disappear completely (e) representing perfect susceptibility or remain full size (f) marking drug resistance.

Referring to FIG. 11, in one embodiment, is an example of a possible visualization of tumor heterogeneity, using a hexagonal grid with cellular groupings for metastatic potential. In this example, metastatic potential or other aspects of a subclone aggressiveness may be represented by varying shades of color. More dangerous subclones obtain darker or more intense coloring. This approach allows more exact depicting of the therapy effectiveness by combining subclones relative sizes, its drug susceptibility, and information about actual danger they pose.

Thus, a combination of FIGS. 9, 10, and 11 results in a possible visualization of tumor heterogeneity, using a hexagonal grid with cellular groupings, that simultaneously depicts relative tumor subclone amounts, medication susceptibility, and metastatic potential.

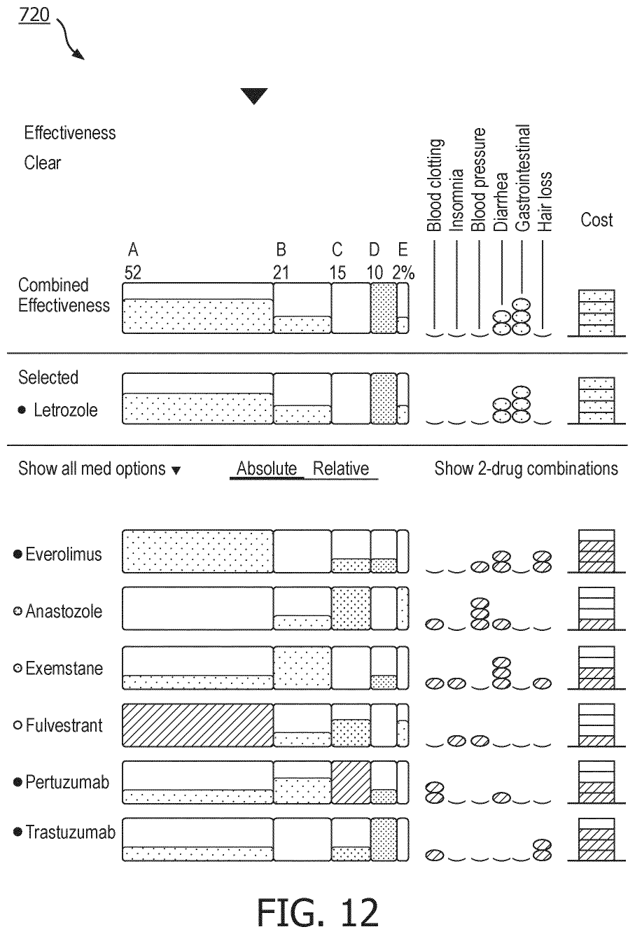

Referring to FIG. 12, in one embodiment, is a larger representation of the drug selection panel 720 of FIG. 7B. The drug selection panel 720 comprises a list of one or more possible therapies that can be utilized to treat one or more of the subclones (Letrozole, Everolimus, Anastozole, etc.). According to an embodiment, the possible therapies are obtained by tumor visualization system 200 as part of the therapy effectiveness information from step 130 of method 100 in FIG. 1.

According to an embodiment, for each one of the one or more possible therapies that can be utilized to treat one or more of the subclones, the drug selection panel 720 shows the predicted effectiveness of the therapy on each of the different subclones. This is similar to the indication of effectiveness shown in the therapy effectiveness panel 710, but shown in a different way (although it may be shown in the same or similar way). In this example of the drug selection panel 720, the indication of effectiveness is shown as a bar graph for each of the different subclones, with a higher bar indicating greater predicted effectiveness (indicating, for example, greater eradication or effect on the subclone cells). For example, the selected therapy (Letrozole) is not effective against subclone C, and therefore the bar graph for subclone C is empty. The therapy is very effective against subclone D and therefore the bar graph for subclone D is full. The therapy works with varying success for subclones A, B, and E as shown by the bar graphs.

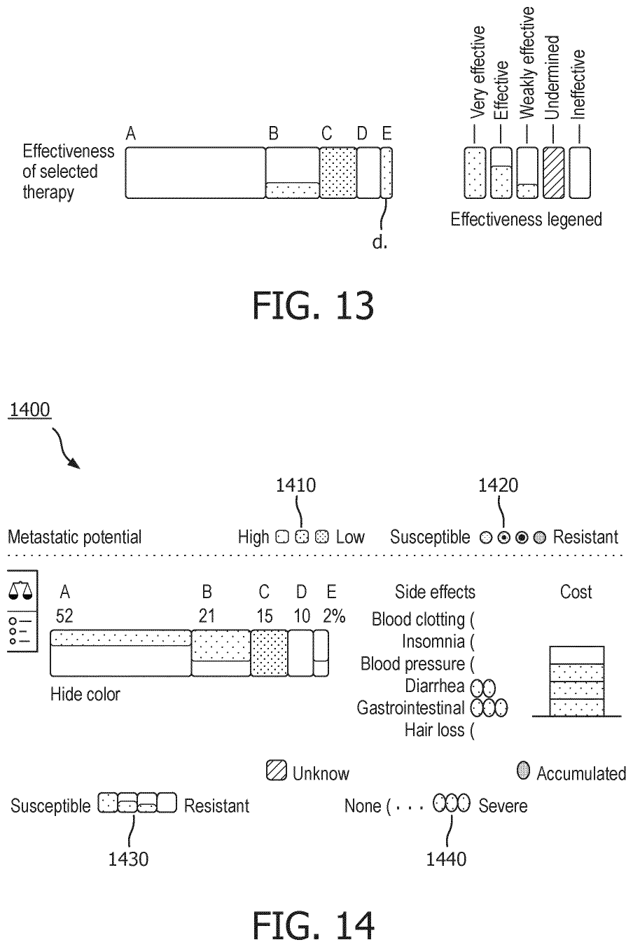

Referring to FIG. 13, in one embodiment, is an example of a possible visualization of the indication of therapy effectiveness, in which a more filled bar or block represents a very effective therapy, and an unfilled bar or block represents a very ineffective therapy. Hatching or another indication could indicate an undetermined effectiveness of the therapy on a subclone represented by a bar or block.

Referring to FIG. 14, in one embodiment, is an optional portion of the therapy effectiveness panel 710 showing a legend 1400. The legend, which can be shown or hidden, shows the legend for metastatic potential 1410, for susceptibility and resistance in 1420 and 1430, and for the severity of side effects 1440.

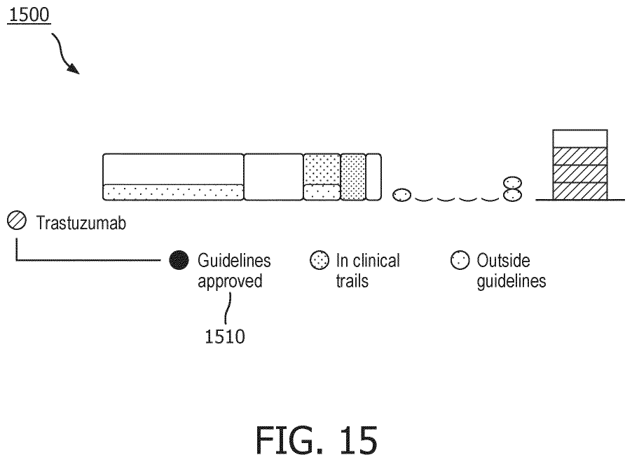

Referring to FIG. 15, in one embodiment, is an optional portion of the drug selection panel 720 showing a legend 1500. The legend, which can be shown or hidden, shows the legend for the clinical status of the therapy, including whether it is approved by the guidelines, in clinical trials, or not approved by the guidelines.

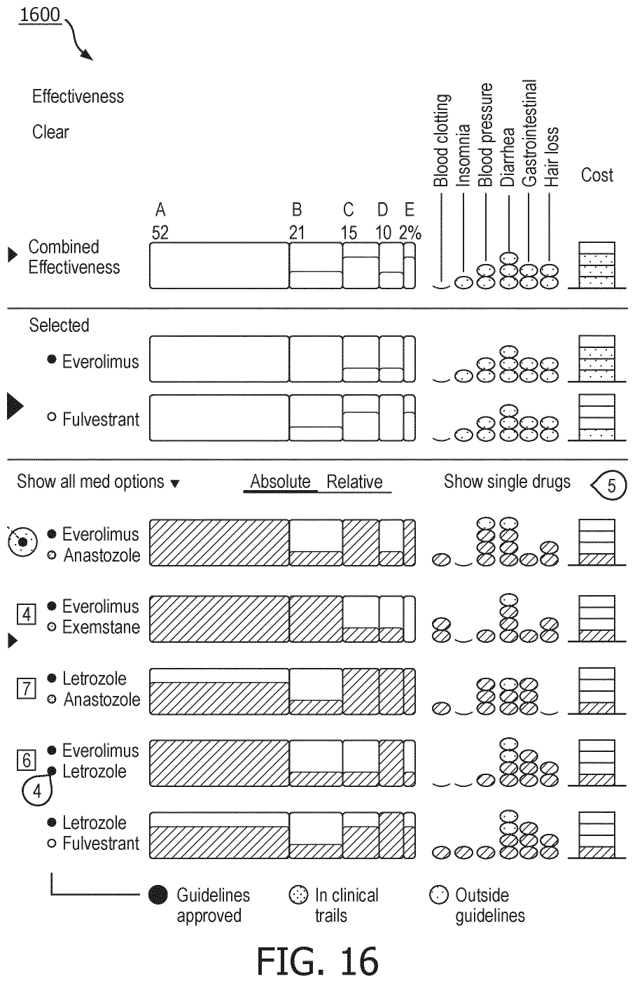

Referring to FIG. 16, in one embodiment, is a larger representation 1600 of a drug selection panel 720 of FIG. 7B. In this embodiment, the drug selection panel 720 comprises a combined effectiveness of two or more therapies on the one or more of the subclones. In this example, the therapies Everolimus and Fulvestrant are selected. The panel shows individual effectiveness for Everolimus and Fulvestrant on the subclones, and shows a combined effectiveness of the two drugs, along with information such as cumulative side effects and cost.

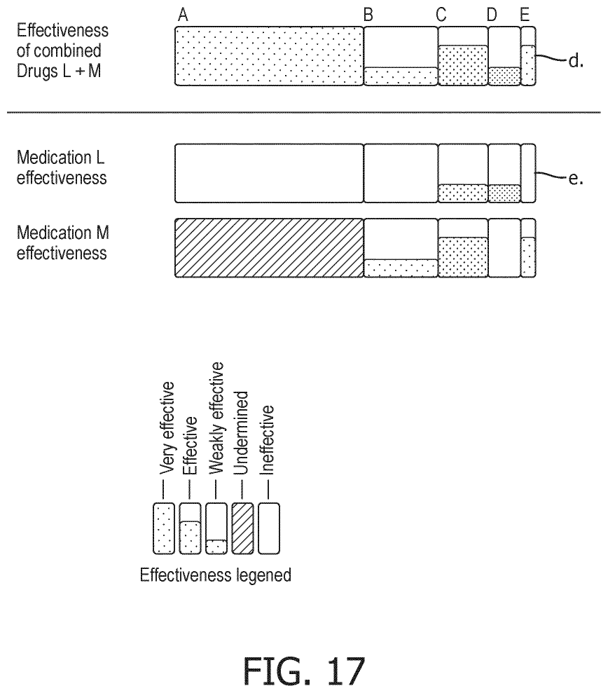

Referring to FIG. 17, in one embodiment, is an example of a possible visualization of combined effectiveness of two or more therapies on the one or more of the subclones. As described above, the size of the dark area within each block represents degree of drug influence on the corresponding subclone (d). The resulting image enables quick assessment of therapy's effect. The less of the block that remains light (not fully affected), the better potential results. Total therapy effectiveness can be shown as a combination of component drug influence. For each subclone merging algorithm promotes strongest drug effect to combine effectiveness representation and disregards the others. The combined drug effect visual merges the subclone blocks into fused representation of tumor susceptibility (dark area) and resistance (light area), while single drug depiction (e) clearly separates subclones to emphasize specific interactions of each drug with each subclone.

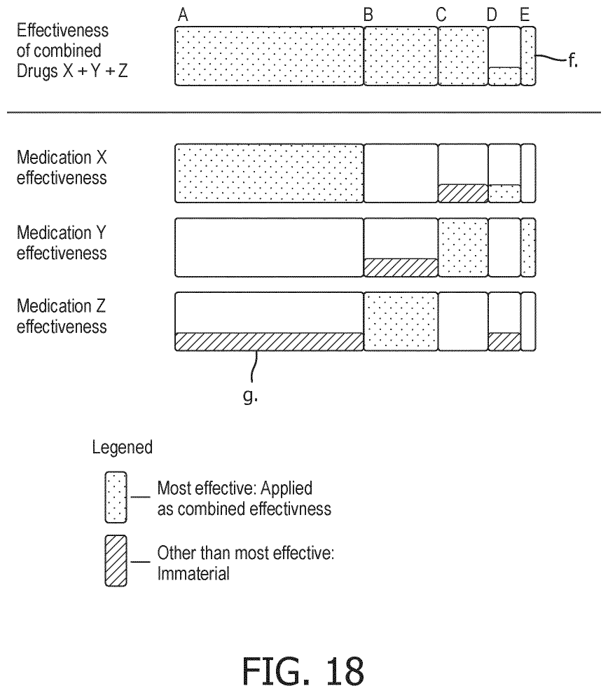

Referring to FIG. 18, in one embodiment, is an example of a possible visualization of combined effectiveness of two or more therapies on the one or more of the subclones. In this embodiment, in order to emphasize which medication contributes to combined effectiveness (f) the strongest subclone-drug interactions is represented in full strength color while the others are grayed-out (g).

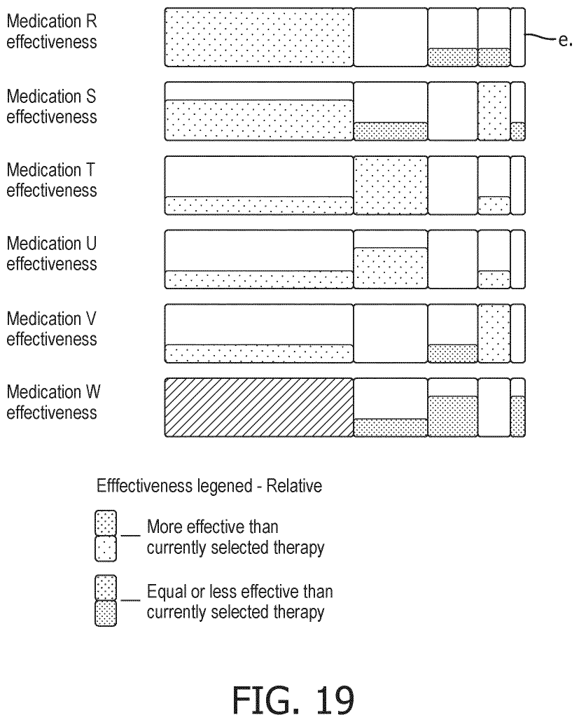

Referring to FIG. 19, in one embodiment, is an example of a possible visualization that assists with further selection of drugs to improve the combined effectiveness of two or more drugs in the selected therapy on the one or more of the subclones. In this embodiment, after an initial proposed therapy is set up (d, in FIG. 17) the list of medications to be considered as possible additions is presented (e). In this list the subclone-drug interactions that are more effective than currently selected therapy are highlighted. The drugs with most beneficial interactions are pushed to the top of the list. The addition of new drug to proposed therapy changes its benefits profile and causes the list of the rest of the drugs to be reordered accordingly.

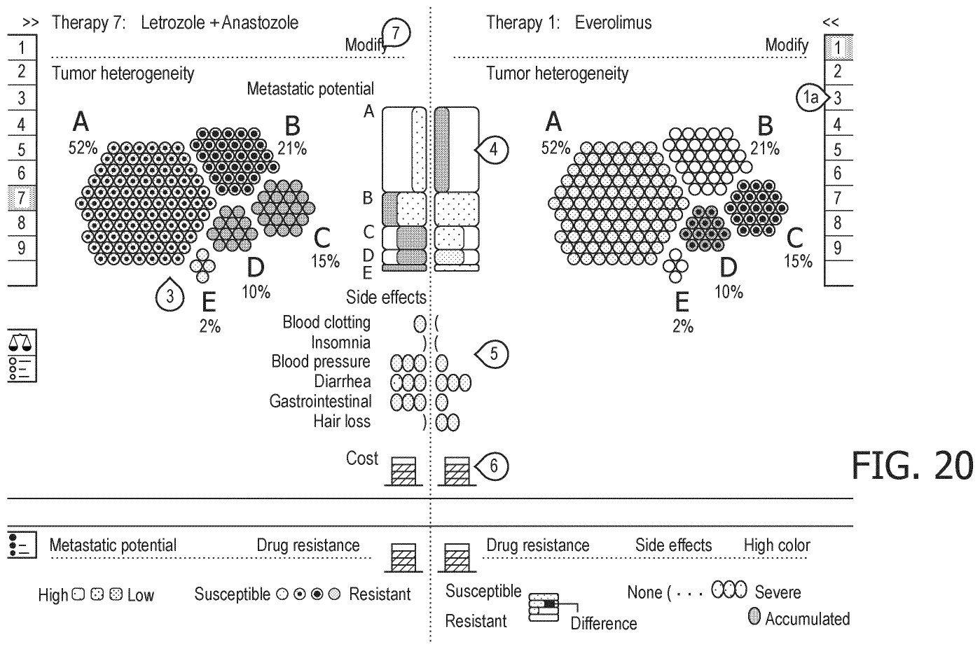

Referring to FIG. 20, in one embodiment, is a visualization 2000 of the therapy effectiveness panel 710 in which two different possible therapies are compared. In this embodiment, the effect of Therapy 1: Everolimus is compared to Therapy 7: Letrozole+Anastozole. The comparison shows the effectiveness of each therapy on the five different subclones in terms of both the bubbles and the bar graphs.

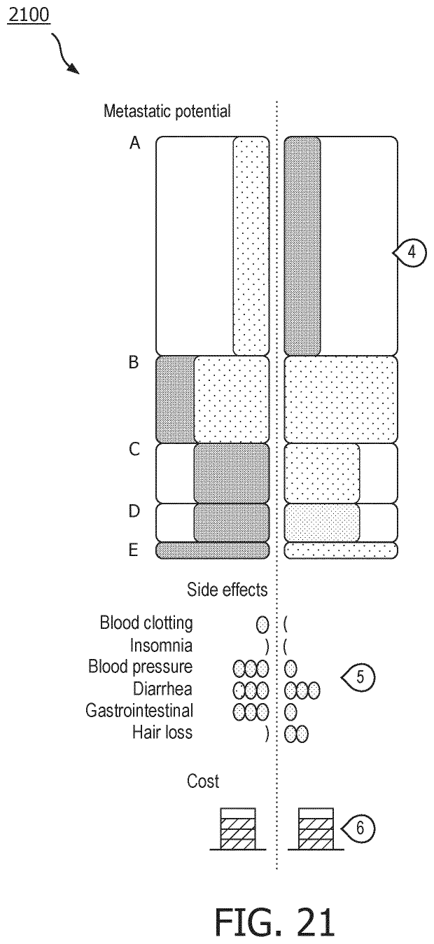

Referring to FIG. 21 is a larger representation 2100 of a portion of the visualization 2000 in FIG. 20. The comparison shows the metastatic potential of the five different subclones in a direct side-by-side alignment of the bar graphs. The comparison also shows side effects and costs in a direct side-by-side alignment, allowing for each of comparison.

According to an embodiment, a user can interactively navigate between different panels, different subclones, different therapies, different combinations of therapies, and other aspects of the embodiments described or otherwise envisioned herein. Navigation can be accomplished using any method of user interface interaction.

Referring to FIG. 2 is a schematic representation of a tumor visualization system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may be located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, the electronic medical record database or system 270 is an electronic medical records database from which the information about the patient and the heterogeneity assessment, among other possible information, may be obtained or received. The electronic medical records database or system may be a local or remote database and is in direct and/or indirect communication with the tumor visualization system 200. Thus, according to an embodiment, the system comprises an electronic medical record database or system 270.

According to another embodiment, heterogeneity assessment is obtained by performing heterogeneity analysis on the data from a LIMS, DICOM and/or EMR system. Thus, the system may comprise or be in communication with a patient imaging data system or database 272, such as an LIMS or DICOM system or database. According to an embodiment, the system comprises or is in communication with a laboratory information management system 276 comprising testing or analysis data, and/or other information. System 200 can receive information from the laboratory information management system 276, and can send information to the laboratory information management system 276. The laboratory information management system 276 may be a local or remote system or database and is in direct and/or indirect communication with the tumor visualization system 200.

According to an embodiment, the therapy database 274 is a database from which information about the effectiveness of one or more therapies for one or more tumors or subclones can be received or obtained. The therapy database may be a local or remote database and is in direct and/or indirect communication with the tumor visualization system 200. Thus, according to an embodiment, the system comprises a therapy database 274.

According to an embodiment, storage 260 of system 200 may store one or more algorithms, modules, and/or instructions to carry out one or more functions or steps of the methods described or otherwise envisioned herein. For example, the system may comprise, among other instructions or data, patient information 262a, patient heterogeneity assessment data and/or instructions 262b, retrieval instructions 263, generation instructions 264, reporting instructions 265, and therapeutic instructions 266.

According to an embodiment, patient information 262a is any information about the patient, and optionally the corresponding heterogeneity assessment, that the tumor visualization system can or may utilize for analysis as described or otherwise envisioned herein. According to an embodiment, the patient information comprises one or more of demographic and clinical information about the patient, an identification of the patient's tumor type, and a heterogeneity assessment of the patient's tumor. Other information is possible.

According to an embodiment, patient heterogeneity assessment data and/or instructions 262b direct the system to perform the heterogeneity assessment, or to retrieve the data necessary for a heterogeneity assessment. According to an embodiment, the heterogeneity assessment of the patient's tumor comprises a qualitative or quantitative assessments of the heterogeneity of the patient's tumor as described or otherwise envisioned herein.

According to an embodiment, the retrieval instructions 263 direct the system to receive, retrieve, or otherwise obtain therapy effectiveness information for each of two or more subclones of the tumor. This therapy effectiveness information is identified and obtained based on the received identification of the patient's tumor type and the received heterogeneity assessment of the patient's tumor. Therapy effectiveness can be based on known effectiveness of one or more therapies for a tumor type, as well as for an identified subclone type. The therapy effectiveness information can be obtained, for example, from therapy database 274.

According to an embodiment, the generation instructions 264 direct the system to generate one or more visual representations described or otherwise envisioned herein. This may be performed immediately after the patient information is received and the therapy effectiveness information in obtained, or may be performed using stored information. The system may generate the one or more visual representations on demand or as a result of programming or other automation. According to an embodiment, the system generates the one or more visual representations in response to a user input. For example, a physician or other healthcare professional may request the information. Alternatively, the system may be partially or entirely automated, such that the one or more visual representations are automatically calculated. For example, the system may be a standalone system or a component of a medical data or analysis system, which may comprise a user interface that displays information about a patient. This information may comprise one or more of the information and graphs described or otherwise envisioned herein.

According to an embodiment, reporting instructions 265 direct the system to provide, via a user interface, the generated one or more visual representations. For example, a visual representation may be displayed to a medical professional or other user, including the patient, via the user interface of the system. The generated one or more visual representations may be provided to a user via any mechanism for display, visualization, or otherwise providing information via a user interface. According to an embodiment, the information may be communicated by wired and/or wireless communication to a user interface and/or to another device. For example, the system may communicate the information to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands. As just one non-limiting example, the user interface may be a component of a patient monitoring system.

According to an embodiment, the reporting instructions 265 direct the system to provide, via the user interface, options for interaction with the provided one or more visual representations. For example, the provided display can comprise input fields or other interactive mechanisms allowing the user to provide input. Accordingly, a user can provide input to the system via the user interface, in response to the information provided via the reporting mechanisms. For example, the clinician can select a therapy to be administered to the patient, which can then be reported to a clinical decision support system or patient management system, optionally together with the evidence of all available options, for future auditing purposes among other things.

According to an embodiment, therapeutic instructions 266 direct the system to manage therapy information. For example, the therapeutic instructions 266 can manipulate the therapy effectiveness information for each of two or more subclones of the tumor, such as by creating and evaluating different combination therapy options. The therapeutic instructions 266 can, for example, evaluate and rank the effectiveness of possible drug combinations on one or more subclones. Therapy effectiveness can be based on known effectiveness of one or more therapies for a tumor type, as well as for an identified subclone type. The therapy effectiveness information can be obtained, for example, from therapy database 274.

Accordingly, within the context of the disclosure herein, aspects of the embodiments may take the form of a computer program product embodied in one or more non-transitory computer readable media having computer readable program code embodied thereon. Thus, according to one embodiment is a non-transitory computer-readable storage medium comprising computer program code instructions which, when executed by a processor, enables the processor to carry out a method including: (i) receiving information about a patient, comprising demographic and clinical information about the patient, an identification of the patient's tumor type, and a heterogeneity assessment of the patient's tumor; (ii) obtaining, based on the identification of the patient's tumor type and the heterogeneity assessment of the patient's tumor, therapy effectiveness information for each of the two or more subclones of the tumor; (iii) generating a visual representation of the heterogeneity assessment of the patient's tumor and the obtained therapy effectiveness information for each of the two or more subclones of the tumor; and (iv) providing, via a user interface of the system, the generated visual representation. The program code may perform entirely on a user's computer, partly on a user's computer, as a stand-alone software package, partly on a user's computer and partly on a remote computer, or entirely on a remote computer or server.

According to an embodiment, the tumor visualization system is configured to process many thousands or millions of datapoints in the analysis of the received patient information, retrieval and analysis of the tumor effectiveness information, in the generation of the one or more visualizations, and for the display of the generated one or more visualizations. For example, analyzing this data, generating these visualizations, and providing the generated visualizations, requires processing of millions of datapoints. This requires millions or billions of calculations, which a human mind could not perform in a lifetime.

By providing improved visualization of tumor heterogeneity and therapy effectiveness, this novel tumor visualization system has an enormous positive effect on patient care compared to prior art systems. Improved understanding of tumor heterogeneity and therapy effectiveness, made entirely possible by the novel visualizations provided herein, improves clinician analysis and thus improves patient wellbeing and care, among other improvements.

### Improved Patient Survival Analysis and Visualization

According to another aspect is a system and method configured to generate and provide a visual representation of survival for a patient. More generally, Applicant has recognized and appreciated that it would be beneficial to provide improved methods and systems for the visualization of patient survival probability over time. Accordingly, a survival statistics analysis system is described which generates and provides a visual representation of survival for a patient. The survival statistics analysis system receives information about a patient, including at least a patient diagnosis and a date of diagnosis. The system calculates, using the patient information and a reference survival dataset, one or more survival statistics for the patient for a first time period relative to a first historical cohort of patients. The system then generates a visual representation of the calculated one or more survival statistics and provides the generated visual representation via a user interface of the system. According to an embodiment, the generated visual representation of survival for a patient comprises one or more of a survival function graph, a median survival graph, a mortality risk graph, and a survival probability graph.

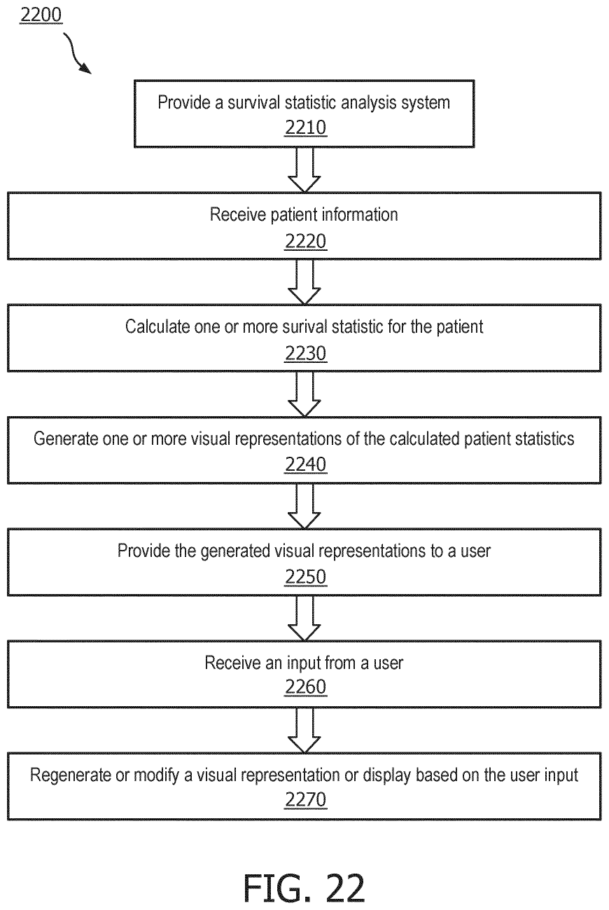

Referring to FIG. 22, in one embodiment is a flowchart of a method 2200 for generating and providing a visual representation of survival for a patient using a survival statistics analysis system. The methods described in connection with the figures are provided as examples only, and shall be understood to not limit the scope of the disclosure. The survival statistics analysis system can be any of the systems described or otherwise envisioned herein. The survival statistics analysis system can be a single system or multiple different systems.

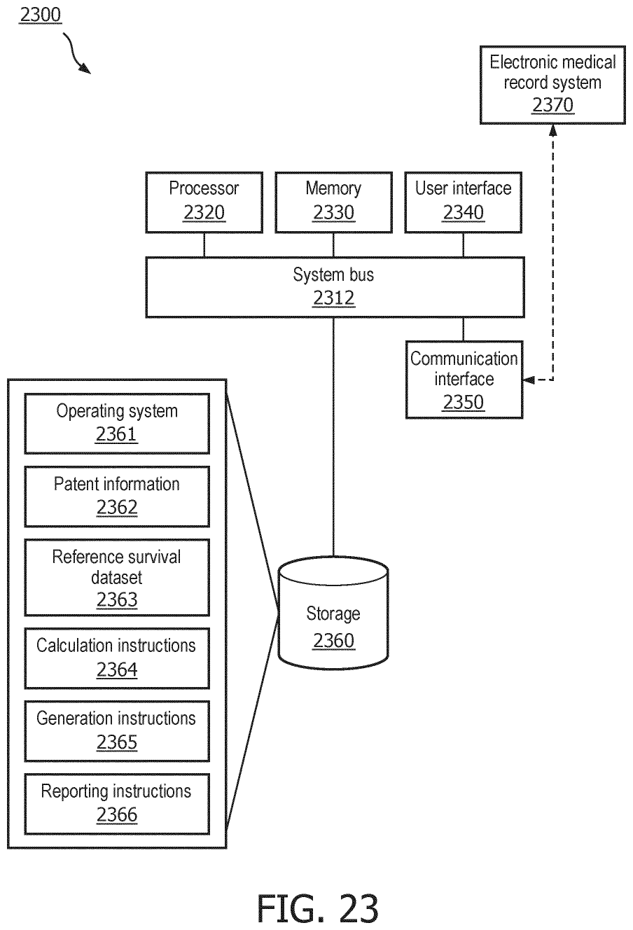

At step 2210 of the method, a survival statistics analysis system 2300 is provided. Referring to an embodiment of a survival statistics analysis system 2300 as depicted in FIG. 23, for example, the system comprises one or more of a processor 2320, memory 2330, user interface 2340, communications interface 2350, and storage 2360, interconnected via one or more system buses 2312. It will be understood that FIG. 23 constitutes, in some respects, an abstraction and that the actual organization of the components of the system 2300 may be different and more complex than illustrated. Additionally, survival statistics analysis system 2300 can be any of the systems described or otherwise envisioned herein. Other elements and components of survival statistics analysis system 2300 are disclosed and/or envisioned elsewhere herein.

At step 2220 of the method, the survival statistics analysis system receives information about a patient. The patient information can be any information about the patient that the survival statistics analysis system can or may utilize for analysis as described or otherwise envisioned herein. According to an embodiment, the patient information comprises one or more of demographic information about the patient, a diagnosis for the patient, medical history of the patient, and/or any other information. For example, demographic information may comprise information about the patient such as name, age, body mass index (BMI), and any other demographic information. The diagnosis for the patient may be any information about a medical diagnosis for the patient, historical and/or current. The medical history of the patient may be any historical admittance or discharge information, historical treatment information, historical diagnosis information, historical exam or imaging information, and/or any other information.

The patient information is received from one or a plurality of different sources. According to an embodiment, the patient information is received from, retrieved from, or otherwise obtained from an electronic medical record (EMR) database or system 2370. The EMR database or system may be local or remote. The EMR database or system may be a component of the survival statistics analysis system, or may be in local and/or remote communication with the survival statistics analysis system. The received patient information may be utilized immediately, or may be stored in local or remote storage for use in further steps of the method.

At step 2230 of the method, the survival statistics analysis system calculates one or more survival statistics for the patient. Although the calculated output is identified herein as a “survival statistic,” it will be understood that the term is non-limiting and means, among other possible definitions, a probability, likelihood, or estimate for the patient including mortality, survival, and/or many other probabilities, likelihoods, or estimates for the patient.

The system may calculate the one or more survival statistics for the patient on demand or as a result of programming or other automation. According to an embodiment, the survival statistics analysis system calculates one or more survival statistics for the patient in response to a user input. For example, a physician or other healthcare professional may request the information. Alternatively, the survival statistics analysis system may be partially or entirely automated, such that survival statistics for the patient are automatically calculated. For example, the system may be a standalone system or a component of a medical data or analysis system, which may comprise a user interface that displays information about a patient. This information may comprise one or more of the survival statistics and graphs described or otherwise envisioned herein.

The one or more survival statistics for the patient are calculated pursuant to any of the methods described or otherwise envisioned herein. For example, described below are several different non-limiting methods and examples for calculating the one or more survival statistics for the patient. These and many other methods may be utilized by the system.

According to an embodiment, the survival statistics analysis system calculates one or more survival statistics for the patient for a predetermined or user-defined first time period. The system may calculate the one or more survival statistics for any time period. Non-limiting examples of the first time period include days, weeks, months, years, and decades. For example, the system may calculate the one or more survival statistics for a time period of five years, which is a commonly-utilized time period for survival data, although the first time period may also be shorter or longer than five years.

According to an embodiment, the survival statistics analysis system comprises or is in communication with a reference survival dataset, which is utilized for the calculation of the one or more survival statistics for the patient. The reference survival dataset can be received from one or a plurality of different sources. According to an embodiment, the reference survival dataset is received from, retrieved from, or otherwise obtained from the electronic medical record (EMR) database or system 2370. The EMR database or system may be local or remote. The EMR database or system may be a component of the survival statistics analysis system, or may be in local and/or remote communication with the survival statistics analysis system. Alternatively, the reference survival dataset may be a remote dataset, or another stored dataset of the system. The received reference survival dataset may be utilized immediately, or may be stored in local or remote storage for use in further steps of the method.

According to an embodiment, the reference survival dataset comprises information about one or more historical patients, which may be collected, filtered, or otherwise combined into one or more patient cohorts. A patient cohort may be, for example, a set of a plurality of historical patients for which survival data is available for at least the first time period. The patient cohort may be comprised of historical patients that have the same diagnosis, conditions, treatment, or other similarities to the patient for which the one or more survival statistics are being calculated. According to an embodiment, therefore, the system identifies a patient cohort relevant to a patient or subject being analyzed. According to another embodiment, the system identifies a patient cohort relevant to the patient or subject being analyzed, as well as to a patient cohort that does not have the same diagnosis, conditions, treatment, or other similarities to the patient for which the one or more survival statistics are being calculated. This later embodiment, for example, may enable analysis or determination of an impact of a diagnosis, conditions, treatment, or other similarities on the patient for which the one or more survival statistics are being calculated.

According to an embodiment, the calculated survival statistics for the patient are utilized immediately, or may be stored in local or remote storage for use in further steps of the method. Thus, for example, stored survival statistics for the patient can be recalled and utilized whenever a display is directed to, or is otherwise designed or programmed to, display patient information.

At step 2240 of the method, the survival statistics analysis system generates one or more visual representations of the calculated one or more survival statistics. This may be performed immediately after the one or more survival statistics are calculated, or may be performed using stored survival statistics. The system may generate the one or more visual representations of the calculated one or more survival statistics on demand or as a result of programming or other automation. According to an embodiment, the survival statistics analysis system generates the one or more visual representations in response to a user input. For example, a physician or other healthcare professional may request the information. Alternatively, the survival statistics analysis system may be partially or entirely automated, such that the one or more visual representations are automatically calculated. For example, the system may be a standalone system or a component of a medical data or analysis system, which may comprise a user interface that displays information about a patient. This information may comprise one or more of the survival statistics and graphs described or otherwise envisioned herein.

The one or more visual representations of the calculated one or more survival statistics are generated pursuant to any of the methods described or otherwise envisioned herein. For example, described below are several different non-limiting methods and examples of visual representations of calculated survival statistics, and thus the system may be designed or programmed to generate such visual representations using known methods and mechanisms for graphing data. These and many other methods may be utilized by the system.

Once generated, the one or more visual representations of the calculated one or more survival statistics may be utilized immediately, or may be stored in local or remote storage for use in further steps of the method.

At step 2250 of the method, the survival statistics analysis system provides, via a user interface, the generated one or more visual representations of the calculated one or more survival statistics. For example, a visual representation may be displayed to a medical professional or other user, including the patient, via the user interface of the system. The generated one or more visual representations may be provided to a user via any mechanism for display, visualization, or otherwise providing information via a user interface. According to an embodiment, the information may be communicated by wired and/or wireless communication to a user interface and/or to another device. For example, the system may communicate the information to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands. As just one non-limiting example, the user interface may be a component of a patient monitoring system.

According to an embodiment, the display may further comprise patient information such as demographic information about the patient, a diagnosis for the patient, medical history of the patient, and/or any other information.

According to an embodiment, as described herein, some or all of the displayed information may be manipulatable, in response to user input provided via the user interface.

The survival statistics analysis system provides, via a user interface, a generated visual representation of the calculated one or more survival statistics, which can be any of a variety of different statistics and visual representations. According to one embodiment, the visual representation comprises a survival function graph. The survival function graph can include a single graph that displays two different survival statistics, preferably overlaid. The first survival statistic of the survival function graph is a plot of a Kaplan-Meier estimate for the first historical cohort for the first time period. A Kaplan-Meier estimate is a statistic used to estimate a survival function over time. In the healthcare setting, the Kaplan-Meier estimate can be a measure of the fraction of patients or subjects living for a certain amount of time after diagnosis or after treatment.

According to an embodiment, the Kaplan-Meier estimate plot in the graph is based on integrated data from multiple Kaplan-Meier type estimators. The survival function may present overall survival data, disease-free survival, or progression-free survival, among other options. The second survival statistic of the survival function graph is a visualization of mortality in the same period, comprising a plot of trailing mortality. This trailing mortality comprises a percentage of mortality of the first historical cohort for a trailing time period, where the trailing time period is a windowed segment of the first time period. The trailing time period can be any time period which is a subset or segment of the first time period. For example, the trailing time period can be weeks, months, or years. According to one embodiment, the trailing time period is six months, although it may be shorter or longer. According to another embodiment, the trailing time period is one year, although it may be shorter or longer. The trailing time period may be predetermined or preprogrammed, or may be determined in whole or in part based on user input received via the user interface.

According to an embodiment, the first historical cohort for this graph, and any of the other graphs, comprises a set of a plurality of historical patients for which survival data is available for at least the first time period, which is collected, filtered, or otherwise retrieved from the reference survival dataset. For example, the first historical cohort may be comprised of historical patients that have the same diagnosis, conditions, treatment, or other similarities to the patient for which the one or more survival statistics are being calculated. As described herein, the reference survival dataset—and therefore the first historical cohort—can be received or generated from one or a plurality of different sources, and can be a component of the survival statistics analysis system, or may be in local and/or remote communication with the survival statistics analysis system, such as a remote dataset, or another stored dataset of the system.

According to an embodiment, the first time period for this graph, and any of the other graphs, comprises any time period for which the survival statistics are desired. According to another embodiment, the time period is five years, although it may be shorter or longer. The time period may be predetermined or preprogrammed, or may be determined in whole or in part based on user input received via the user interface.

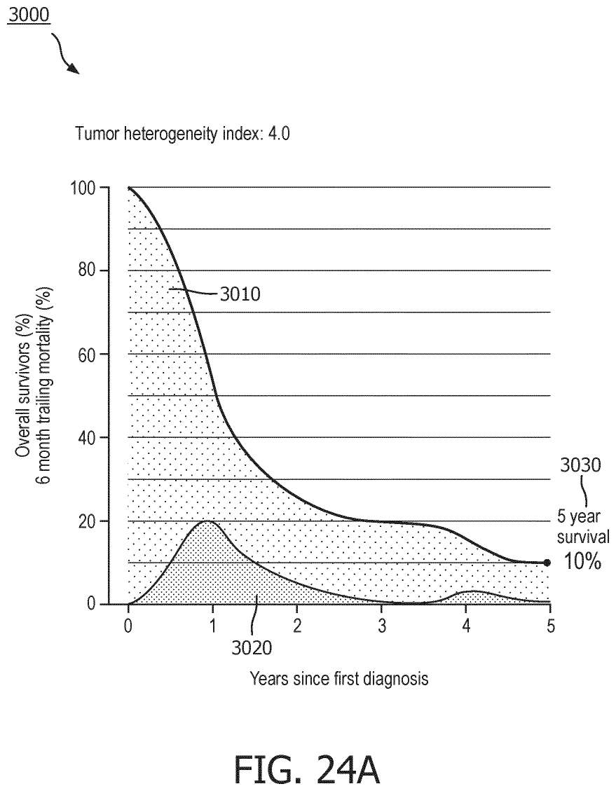

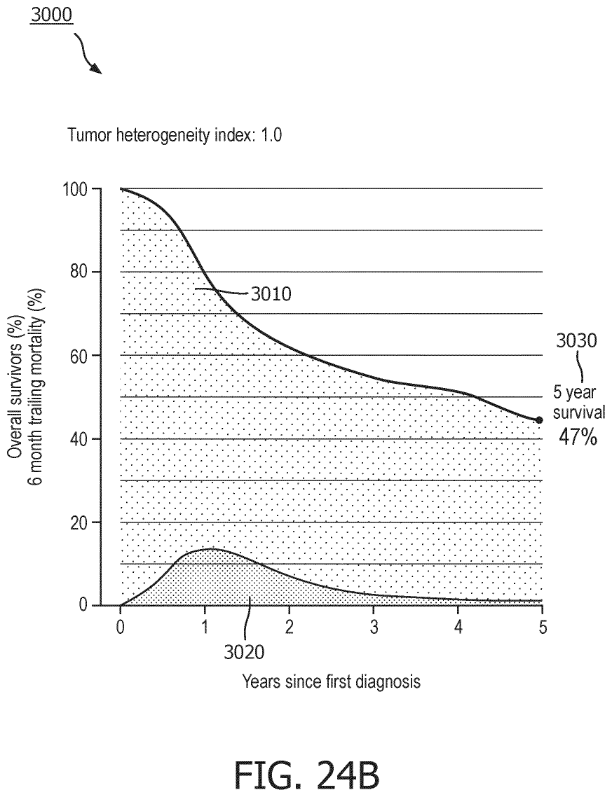

Referring to FIGS. 24A and 24B are survival function graphs 3000. Each survival function graph comprises survival statistics for five years since initial diagnosis, although as described herein this is a non-limiting option. The timeframe may be longer or shorter, and the starting point may be initial diagnosis, treatment, or any other determined starting event. Each survival function graph comprises a Kaplan-Meier estimate 3010 and a trailing mortality plot 3020. To emphasize the survival chances for the entire time period (in this case five years) the last point on the survival function is a final survival percentage 3030, which in this example is a 5-year survival percentage of 10%.

Referring to FIG. 24A, the graph comprises, in this embodiment, a first historical cohort with a particular determined tumor heterogeneity index of 4.0, which indicates a higher level of heterogeneity of cells within the sampled tumor (compared to an index of 1.0, 2.0, 3.0, and variations thereof, for example). In this example, the higher heterogeneity index is correlated with lower survival odds including a 5-year survival percentage of 10%. Referring to FIG. 24B, the graph comprises, in this embodiment, a first historical cohort with a particular determined tumor heterogeneity index of 1.0, which indicates a lower level of heterogeneity of cells within the sampled tumor. In this example, the higher heterogeneity index is correlated with higher survival odds including a 5-year survival percentage of 47%.

According to another embodiment, the visual representation comprises a median survival graph. The median survival graph can include a single graph that displays several different pieces of information, preferably overlaid. The first information provided by the graph is an identification of a median survival time for the first historical cohort for the first time period. The second piece of information is a plot of a percentage of mortality of the first historical cohort for a pre-median survival time period, which will be displayed to the left side of the median survival time. The third piece of information is a plot of a percentage of survival of the first historical cohort for a post-median survival time period, which will be displayed to the right side of the median survival time.

According to an embodiment, this novel method of representing survival time focuses attention on median survival time. For an untrained user, such as a patient, this approach helps recognize the cohort's average life expectancy and how likely it is for death to occur around group average time.

According to an embodiment, the graph is split into two distinct sections using different functions for the period before and after the median survival time (MST). In the interval preceding MST chart tracks the mortality rate as a percentage of overall cohort. In the period following MST the graph uses survival function-number of survivors as a percentage of overall cohort. This type of compound visual concentrates attention on the average survival time. Second important benefit of this approach is the clear depiction of the probability of decease around MST represented by steepness of ‘median peak’.

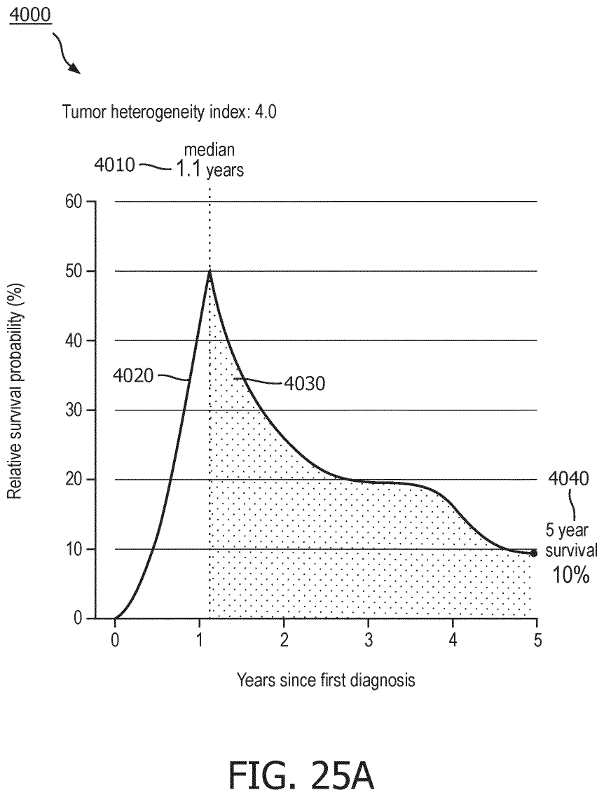

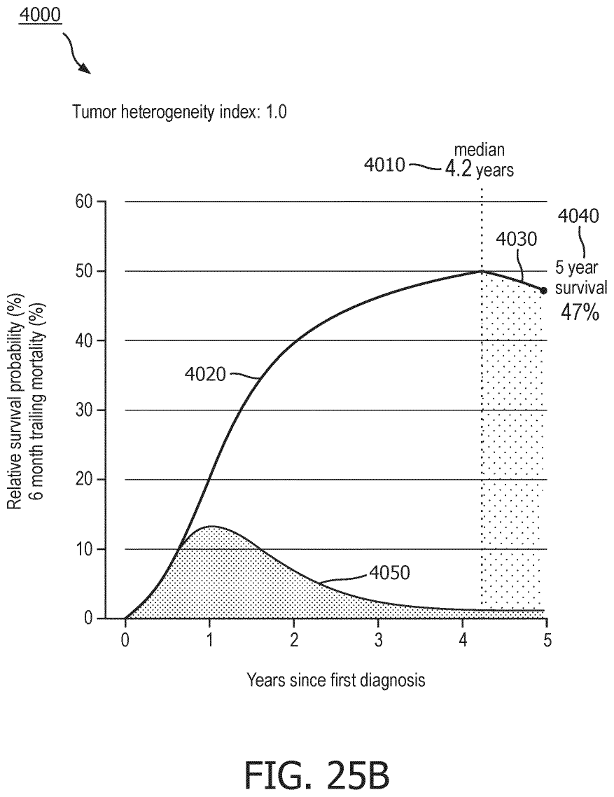

Referring to FIGS. 25A and 25B are median survival graphs 4000. Each median survival graph comprises survival statistics for five years since initial diagnosis, although as described herein this is a non-limiting option. The timeframe may be longer or shorter, and the starting point may be initial diagnosis, treatment, or any other determined starting event. Each median survival graph comprises a median survival time 4010, a pre-median plot of the mortality rate as a percentage of overall cohort 4020, and a post-median plot of the number of survivors as a percentage of overall cohort 4030. To emphasize the survival chances for the entire time period (in this case five years) the last point on the survival function is a final survival percentage 4040, which in this example is a 5-year survival percentage of 10%. The graphs illustrate how deaths can be concentrated in the period around MST giving the average number more significance (THI=4.0 graph in FIG. 25A) or more spread out (THI=1.0 in FIG. 25B) diminishing its predictive value.

Referring to FIG. 25B, to provide additional information the visual may be layered with a mortality graph 4050 showing mortality in a trailing period as the percentage of the original cohort. For example, the trailing period in this graph is 6 months, although the trailing period may be shorter or longer.

Referring to FIG. 25A, the graph comprises, in this embodiment, a first historical cohort with a particular determined tumor heterogeneity index of 4.0, which indicates a higher level of heterogeneity of cells within the sampled tumor. In this example, the higher heterogeneity index is correlated with lower survival odds including a 5-year survival percentage of 10%. Referring to FIG. 25B, the graph comprises, in this embodiment, a first historical cohort with a particular determined tumor heterogeneity index of 1.0, which indicates a lower level of heterogeneity of cells within the sampled tumor. In this example, the higher heterogeneity index is correlated with higher survival odds including a 5-year survival percentage of 47.

According to another embodiment, the visual representation comprises a mortality risk graph. The mortality risk graph can include a single graph that displays a plot of trailing mortality. The plot of trailing mortality comprises a percentage of mortality, for a trailing time period, of the first historical cohort relative to a number of survivors of the first historical cohort at a beginning of the trailing time period, where the trailing time period is a windowed segment of the first time period. The trailing time period can be any time period which is a subset or segment of the first time period. For example, the trailing time period can be weeks, months, or years. According to one embodiment, the trailing time period is six months, although it may be shorter or longer. According to another embodiment, the trailing time period is one year, although it may be shorter or longer. The trailing time period may be predetermined or preprogrammed, or may be determined in whole or in part based on user input received via the user interface.

According to an embodiment, method of calculating mortality reveals changes in mortality risk over a period of time since a triggering event. The period of time may be any period of time, and the triggering event may be any triggering event, including but not limited to a diagnosis, treatment, and other events. According to an embodiment, the risk of non-survival is calculated by representing the number of deaths as a percentage of number of survivors in each time period. This view of data reveals what time periods in the total time period since the triggering event are the most dangerous.

According to one embodiment, the data for the mortality risk graph is calculated using the following equation:

$$Mr = D ( time ) / S \qquad ( Eq . 1 )$$

where Mr is mortality risk, D is the number of deaths during the trailing time period (time), and S is the number of survivors at the beginning of the time period.

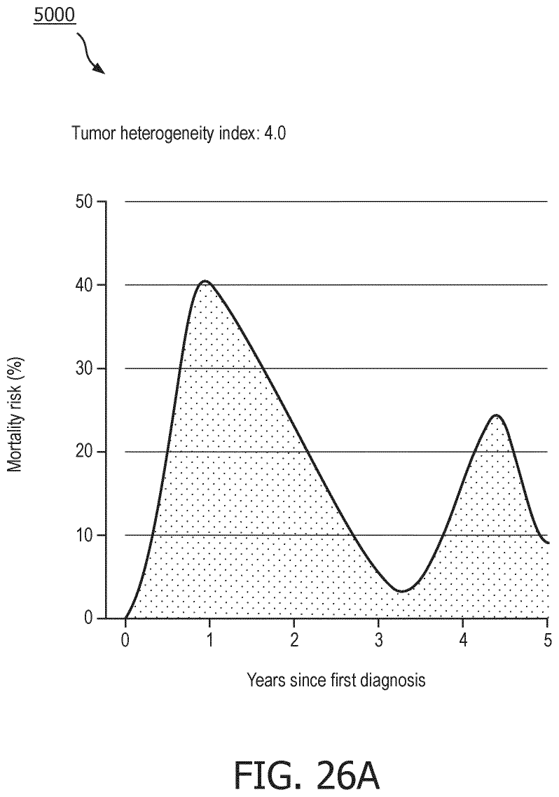

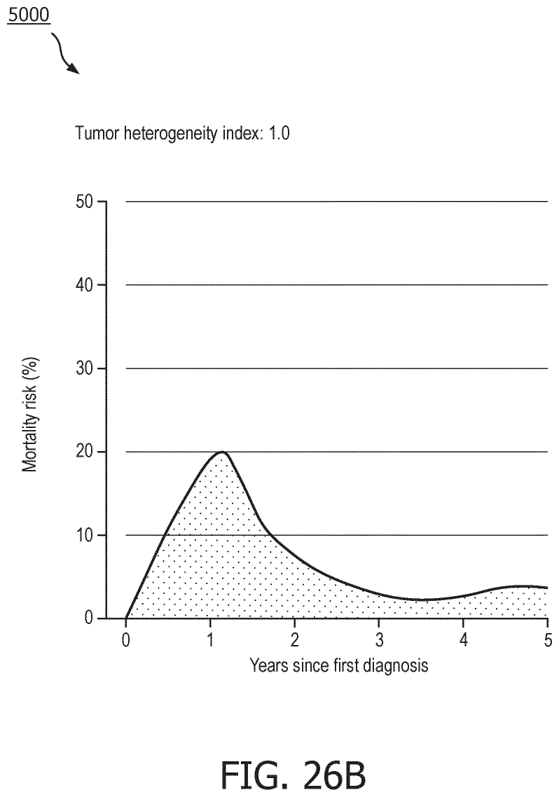

Referring to FIGS. 26A and 26B are mortality risk graphs 5000. Each mortality risk graph comprises survival statistics for five years since initial diagnosis, although as described herein this is a non-limiting option. The timeframe may be longer or shorter, and the starting point may be initial diagnosis, treatment, or any other determined starting event. Referring to FIG. 26A, the graph comprises, in this embodiment, a first historical cohort with a particular determined tumor heterogeneity index of 4.0, which indicates a higher level of heterogeneity of cells within the sampled tumor. Referring to FIG. 26B, the graph comprises, in this embodiment, a first historical cohort with a particular determined tumor heterogeneity index of 1.0, which indicates a lower level of heterogeneity of cells within the sampled tumor.

According to another embodiment, the visual representation comprises a survival probability graph. The survival probability graph includes a plot of survival probability over the first time period. The survival probability is calculated for each of a plurality of time points over the first time period by dividing a total number of survivors of the first historical cohort at the end of the first time period by a total number of survivors of the first historical cohort at the time point being calculated.

According to an embodiment, this view focuses on positive aspects of moving along the survival probability timeline. It illustrates the change in prospects of patients that have already moved forward from diagnosis time (or another triggering event). According to an embodiment, the probability of survival till the end of the time period (such as five years) is calculated by comparing the number of overall survivors of the total time interval to the number of survivors at each represented time point. This view of data, when combined with marker representing time that passed since patient diagnosis, may help in visualizing the hope in the upcoming period.

According to one embodiment, the data for the survival probability graph is calculated using the following equation:

$$Probability of survival ( time ) = S ( time ) / S ( moment ) \qquad ( Eq . 2 )$$

where S(time) is the number of survivors at the end of the total time period (time) and S(moment) is the total number of survivors at the moment in time along the graph.

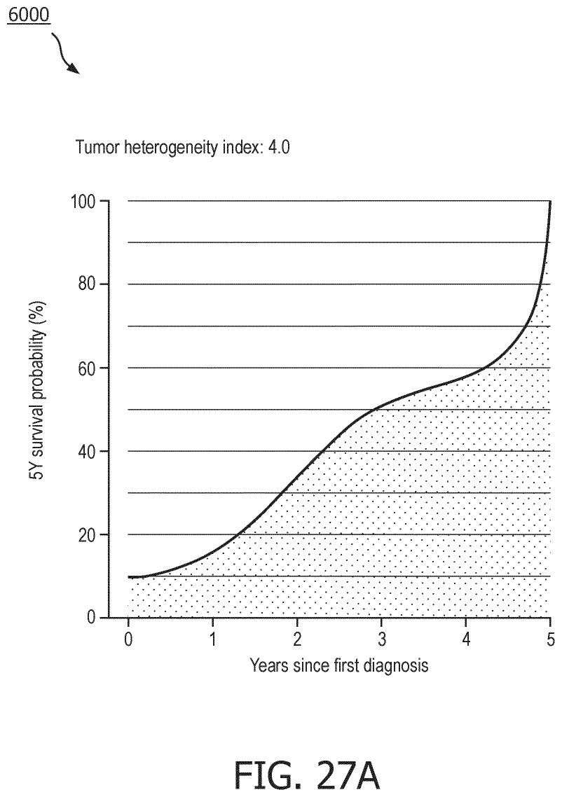

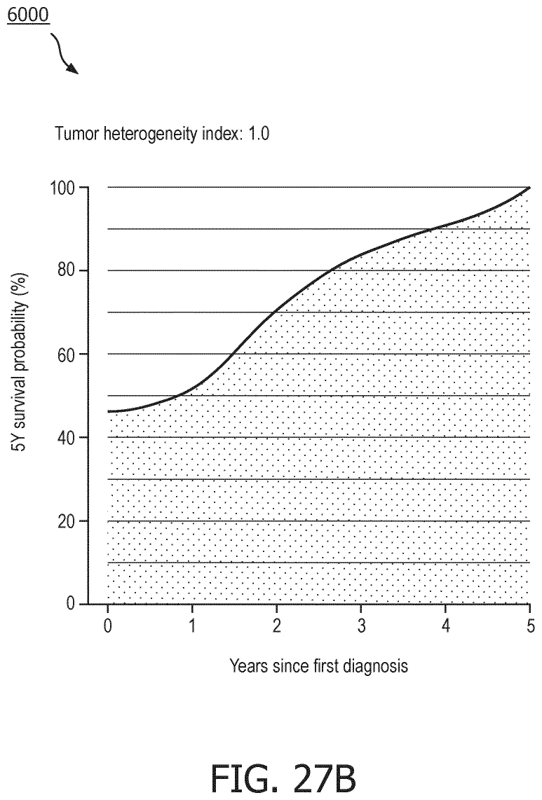

Referring to FIGS. 27A and 27B are survival probability graphs 6000. Each survival probability graph comprises survival statistics for five years since initial diagnosis, although as described herein this is a non-limiting option. The timeframe may be longer or shorter, and the starting point may be initial diagnosis, treatment, or any other determined starting event. Referring to FIG. 27A, the graph comprises, in this embodiment, a first historical cohort with a particular determined tumor heterogeneity index of 4.0, which indicates a higher level of heterogeneity of cells within the sampled tumor. Referring to FIG. 27B, the graph comprises, in this embodiment, a first historical cohort with a particular determined tumor heterogeneity index of 1.0, which indicates a lower level of heterogeneity of cells within the sampled tumor.

At step 2260 of the method, a user provides input about the displayed one or more visualizations via the user interface of the survival statistics analysis system 2000. The input can be any input, including but not limited to a command to modify a visualization, to add a visualization, to remove a visualization, and/or any other input or command. For example, the user interface may be configured to allow a user to navigate between two or more visualizations. The user interface may be configured to display one, two, or more visualizations at one time. The user may be a healthcare professional, the patient, and/or any other user.

Thus, at step 2270 of the method, the survival statistics analysis system regenerates one or more visualizations based on the received input, such as returning to a previous step of the method and generating the visualization based on the input. Alternatively, the system may modify a visualization based on the received input. As just one example, the user may adjust one or more of the first historical cohort, the first time period, and a trailing time period for one or more visualizations. This will necessitate that the system recalculate and/or regenerate the visualizations, which can then be displayed via the user interface. Many other modifications are possible.

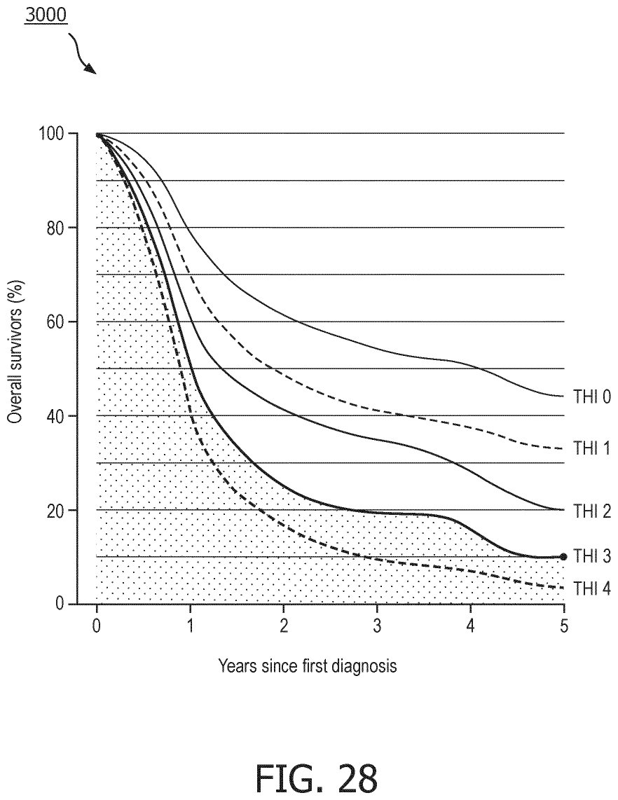

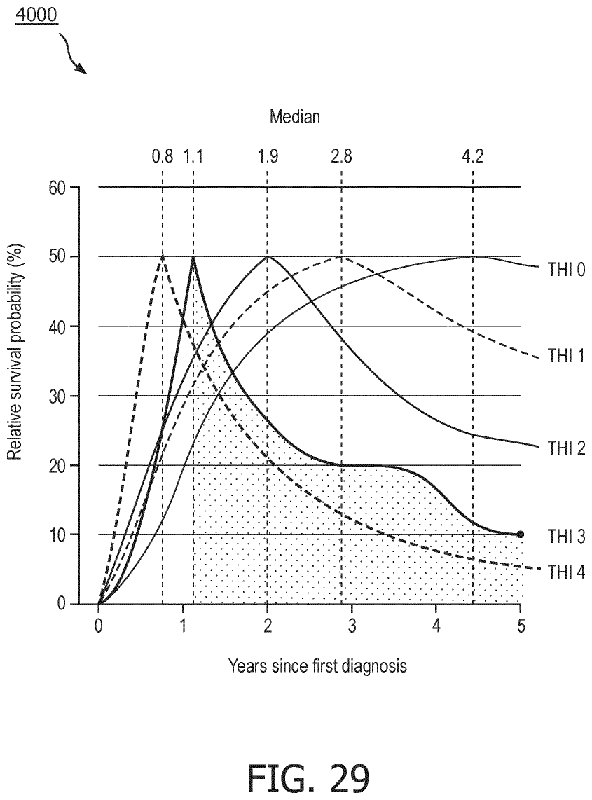

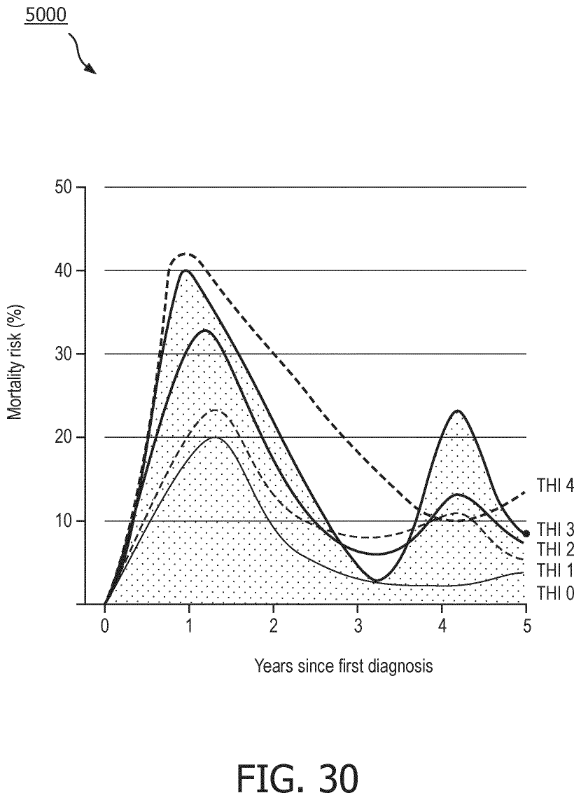

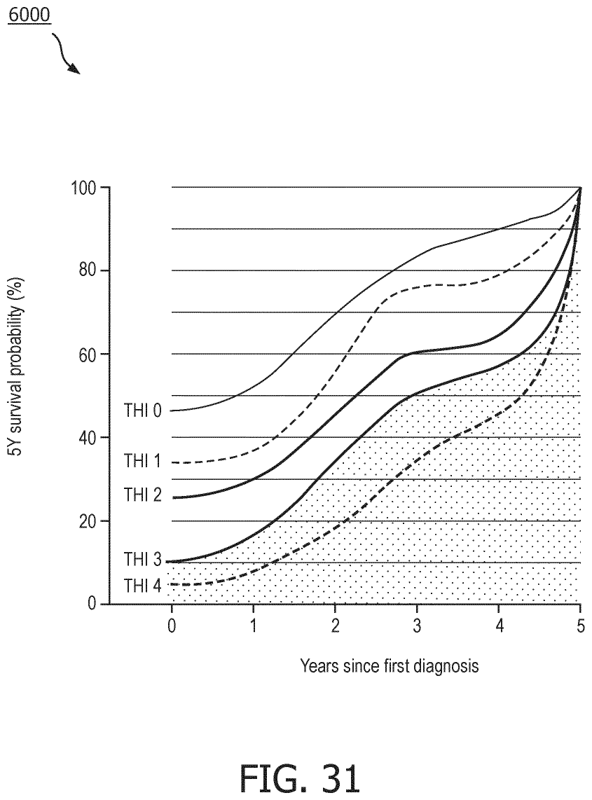

According to an embodiment, any one of the graphs described above or otherwise envisioned herein may comprise a plurality of plots, each calculated with a different historical cohort. For example, the different survival function plots in graph 3000 in FIG. 28 each comprise a different historical cohort each having a different tumor heterogeneity index (THI). As another example, the different median survival graphs 4000 in FIG. 29 each comprise a different historical cohort each having a different THI. As another example, the different mortality risk graphs 5000 in FIG. 30 each comprise a different historical cohort each having a different THI. As another example, the different survival probability graphs 6000 in FIG. 31 each comprise a different historical cohort each having a different THI.

Referring to FIG. 23 is a schematic representation of a survival statistics analysis system 2300. System 2300 may be any of the systems described or otherwise envisioned herein, and may comprise any of the components described or otherwise envisioned herein. It will be understood that FIG. 23 constitutes, in some respects, an abstraction and that the actual organization of the components of the system 2300 may be different and more complex than illustrated.

According to an embodiment, system 2300 comprises a processor 2320 capable of executing instructions stored in memory 2330 or storage 2360 or otherwise processing data to, for example, perform one or more steps of the method. Processor 2320 may be formed of one or multiple modules. Processor 2320 may take any suitable form, including but not limited to a microprocessor, microcontroller, multiple microcontrollers, circuitry, field programmable gate array (FPGA), application-specific integrated circuit (ASIC), a single processor, or plural processors.

Memory 2330 can take any suitable form, including a non-volatile memory and/or RAM. The memory 2330 may include various memories such as, for example L1, L2, or L3 cache or system memory. As such, the memory 2330 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 2300. It will be apparent that, in embodiments where the processor implements one or more of the functions described herein in hardware, the software described as corresponding to such functionality in other embodiments may be omitted.

User interface 2340 may include one or more devices for enabling communication with a user. The user interface can be any device or system that allows information to be conveyed and/or received, and may include a display, a mouse, and/or a keyboard for receiving user commands. In some embodiments, user interface 2340 may include a command line interface or graphical user interface that may be presented to a remote terminal via communication interface 2350. The user interface may be located with one or more other components of the system, or may located remote from the system and in communication via a wired and/or wireless communications network.

Communication interface 2350 may include one or more devices for enabling communication with other hardware devices. For example, communication interface 2350 may include a network interface card (NIC) configured to communicate according to the Ethernet protocol. Additionally, communication interface 2350 may implement a TCP/IP stack for communication according to the TCP/IP protocols. Various alternative or additional hardware or configurations for communication interface 2350 will be apparent.

Storage 2360 may include one or more machine-readable storage media such as read-only memory (ROM), random-access memory (RAM), magnetic disk storage media, optical storage media, flash-memory devices, or similar storage media. In various embodiments, storage 2360 may store instructions for execution by processor 2320 or data upon which processor 2320 may operate. For example, storage 2360 may store an operating system 2361 for controlling various operations of system 2300.

It will be apparent that various information described as stored in storage 2360 may be additionally or alternatively stored in memory 2330. In this respect, memory 2330 may also be considered to constitute a storage device and storage 2360 may be considered a memory. Various other arrangements will be apparent. Further, memory 2330 and storage 2360 may both be considered to be non-transitory machine-readable media. As used herein, the term non-transitory will be understood to exclude transitory signals but to include all forms of storage, including both volatile and non-volatile memories.

While system 2300 is shown as including one of each described component, the various components may be duplicated in various embodiments. For example, processor 2320 may include multiple microprocessors that are configured to independently execute the methods described herein or are configured to perform steps or subroutines of the methods described herein such that the multiple processors cooperate to achieve the functionality described herein. Further, where one or more components of system 2300 is implemented in a cloud computing system, the various hardware components may belong to separate physical systems. For example, processor 2320 may include a first processor in a first server and a second processor in a second server. Many other variations and configurations are possible.

According to an embodiment, the electronic medical record system 2370 is an electronic medical records database from which the information about the patient, and/or the reference survival dataset, may be obtained or received. The electronic medical records database may be a local or remote database and is in direct and/or indirect communication with the survival statistics analysis system 2300. Thus, according to an embodiment, the system comprises an electronic medical record database or system 2370.

According to an embodiment, storage 2360 of system 2300 may store one or more algorithms, modules, and/or instructions to carry out one or more functions or steps of the methods described or otherwise envisioned herein. For example, the system may comprise, among other instructions or data, patient information 2362, reference dataset 2363, calculation instructions 2364, generation instructions 2365, and/or reporting instructions 2366.

According to an embodiment, patient information 2362 is any information about the patient that the survival statistics analysis system can or may utilize for analysis as described or otherwise envisioned herein. According to an embodiment, the patient information comprises one or more of demographic information about the patient, a diagnosis for the patient, medical history of the patient, and/or any other information. For example, demographic information may comprise information about the patient such as name, age, body mass index (BMI), and any other demographic information. The diagnosis for the patient may be any information about a medical diagnosis for the patient, historical and/or current. The medical history of the patient may be any historical admittance or discharge information, historical treatment information, historical diagnosis information, historical exam or imaging information, and/or any other information. The patient information may be stored in storage 2360 of system 2300, and/or may be stored in the EMR database or system 2370, among other options.

According to an embodiment, the reference survival dataset 2363 comprises information about one or more historical patients, which may be collected, filtered, or otherwise combined into one or more patient cohorts. A patient cohort may be, for example, a set of a plurality of historical patients for which survival data is available for at least the first time period. The patient cohort may be comprised of historical patients that have the same diagnosis, conditions, treatment, or other similarities to the patient for which the one or more survival statistics are being calculated. The reference survival dataset 2363 may be stored in storage 2360 of system 2300, and/or may be stored in the EMR database or system 2370, among other options.

According to an embodiment, calculation instructions 2364 direct the system to calculate one or more survival statistics for the patient. The system may calculate the one or more survival statistics for the patient on demand or as a result of programming or other automation. The one or more survival statistics for the patient are calculated pursuant to any of the methods described or otherwise envisioned herein. For example, described herein are several different non-limiting methods and examples for calculating the one or more survival statistics for the patient. These and many other methods may be utilized by the system.

According to an embodiment, generation instructions 2365 direct the system to generate one or more visual representations of the calculated one or more survival statistics. This may be performed immediately after the one or more survival statistics are calculated, or may be performed using stored survival statistics. The system may generate the one or more visual representations of the calculated one or more survival statistics on demand or as a result of programming or other automation. According to an embodiment, the survival statistics analysis system generates the one or more visual representations in response to a user input. For example, a physician or other healthcare professional may request the information. Alternatively, the survival statistics analysis system may be partially or entirely automated, such that the one or more visual representations are automatically calculated. For example, the system may be a standalone system or a component of a medical data or analysis system, which may comprise a user interface that displays information about a patient. This information may comprise one or more of the survival statistics and graphs described or otherwise envisioned herein.

According to an embodiment, reporting instructions 2366 direct the system to provide, via a user interface, the generated one or more visual representations of the calculated one or more survival statistics. For example, a visual representation may be displayed to a medical professional or other user, including the patient, via the user interface of the system. The generated one or more visual representations may be provided to a user via any mechanism for display, visualization, or otherwise providing information via a user interface. According to an embodiment, the information may be communicated by wired and/or wireless communication to a user interface and/or to another device. For example, the system may communicate the information to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands. As just one non-limiting example, the user interface may be a component of a patient monitoring system.

Accordingly, within the context of the disclosure herein, aspects of the embodiments may take the form of a computer program product embodied in one or more non-transitory computer readable media having computer readable program code embodied thereon. Thus, according to one embodiment is a non-transitory computer-readable storage medium comprising computer program code instructions which, when executed by a processor, enables the processor to carry out a method including: (i) receiving information about the patient, comprising at least a patient diagnosis and a date of diagnosis; (ii) calculating, using the patient information and a reference survival dataset, one or more survival statistics for the patient for a first time period relative to a first historical cohort of patients; (iii) generating a visual representation of the calculated one or more survival statistics; and (iv) providing, via a user interface of the system, the generated visual representation. The program code may perform entirely on a user's computer, partly on a user's computer, as a stand-alone software package, partly on a user's computer and partly on a remote computer, or entirely on a remote computer or server.

According to an embodiment, the survival statistics analysis system is configured to process many thousands or millions of datapoints in the calculation of the one or more survival statistics for the patient for the first time period, and in the generation of the one or more visualizations of the calculated one or more survival statistics for the patient, and in the display of the generated one or more visualizations. For example, calculating statistics using data from 100s or 1000s of patients in the historical cohort, and then plotting those calculated statistics, requires processing of millions of datapoints. This requires millions or billions of calculations, which a human mind could not perform in a lifetime.

In addition, the survival statistics analysis system can be configured to continually receive new data for the reference survival dataset. The system can then update the calculations, the visualizations, and the display. This requires the analysis of thousands or millions of datapoints on a continual basis to optimize the display, requiring a volume of calculation and analysis that a human brain cannot accomplish in a lifetime.

By providing improved visualization of survival and mortality statistics, this novel survival statistics analysis system has an enormous positive effect on patient care compared to prior art systems. Improved understanding of survival and mortality statistics, facilitated by the novel visualizations provided herein, can foster improved patient wellbeing and care, among other improvements.

The present invention may be a system, a method, and/or a computer program product. The computer program product may include a non-transitory computer readable storage medium (or media) having computer readable program instructions thereon for causing a system or processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any combination of the foregoing, among other possibilities. Computer readable program instructions described herein can be downloaded to respective computing/processing devices from a computer readable storage medium or to an external computer or external storage device via a network, for example, the internet, a local area network, and/or a wireless network. Aspects of the present invention are described herein with reference to flowchart illustrations and/or block diagrams of methods, apparatus, systems, and computer program products according to embodiments of the invention. It will be understood that each block of the flowchart illustrations and/or block diagrams, and combinations of blocks in the flowchart illustrations and/or block diagrams, can be implemented by computer readable program instructions.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for visualizing heterogeneity of a patient's tumor and a therapy effectiveness for the patient's heterogenous tumor, comprising:
receiving information about a patient, the received information comprising demographic and clinical information about the patient, an identification of the patient's tumor type, and a heterogeneity assessment of the patient's tumor, the heterogeneity assessment comprising an analysis of heterogeneity of the patient's tumor and an identification of two or more subclones of the tumor;
obtaining, based on the identification of the patient's tumor type and the heterogeneity assessment of the patient's tumor, therapy effectiveness information for each of the two or more subclones of the tumor;
generating a visual representation of the heterogeneity assessment of the patient's tumor and the obtained therapy effectiveness information for each of the two or more subclones of the tumor; and
providing, via a user interface of the system, the generated visual representation, wherein the visual representation comprises:
1. a tumor heterogeneity graph, comprising: (i) an identification of each of the two or more subclones of the patient's tumor, wherein the identification comprises an indication of a relative amount of each subclone in the patient's tumor; and (ii) a heterogeneity score indicating an overall complexity of tumor composition of the patient, wherein the heterogeneity score is visualized in an ordered data plot of heterogeneity scores of a same tumor type from a patient cohort, indicating a degree of tumor heterogeneity of the patient as a percentile rank within the patient cohort; and
2. a therapy effectiveness graph, comprising an indication of effectiveness of each of a plurality of different therapies for each of the two or more subclones of the patient's tumor.

2. The method of claim 1, wherein the generated visual representation further comprises some or all of the demographic and clinical information about the patient.

3. The method of claim 1, wherein the tumor heterogeneity graph further comprises an indication of a metastatic potential of each subclone in the patient's tumor.

4. The method of claim 1, wherein the two or more subclones are differentiated by one or more molecular and/or histological variations.

5. The method of claim 4, wherein the tumor heterogeneity graph further comprises an identification of some or all of the one or more molecular and/or histological variations specific to each of the two or more subclones.

6. The method of claim 1, wherein each of the two or more subclones of the patient's tumor on the tumor heterogeneity graph are selectable, and wherein selecting a subclone results in a visual display of some or all of the one or more molecular and/or histological variations specific to the selected subclone.

7. The method of claim 1, wherein the therapy effectiveness graph further comprises an indication of effectiveness of a combination of two or more therapies for the two or more subclones of the patient's tumor.

8. The method of claim 1, wherein the tumor heterogeneity graph and/or the therapy effectiveness graph further comprises one or more of side effect information for each of the plurality of different therapies and a cost of each of the plurality of different therapies.

9. The method of claim 1, further comprising the step of receiving, from a user via a user interface, a selection of one or more of the plurality of different therapies to be administered to the patient, wherein the selected one or more of the plurality of different therapies is optionally reported to a clinical decision support system.

10. The method of claim 1, further comprising:
calculating using the received patient information and a reference survival dataset, one or more survival statistics for the patient for a first time period relative to a first historical cohort of patients;
generating a visual representation of the calculated one or more survival statistics; and
providing, via a user interface of the system, the generated visual representation, wherein the one or more survival statistics comprises one or more of:
1. a survival function graph, the survival function graph comprising a single graph with both: (i) a plot of a Kaplan-Meier estimate for the first historical cohort for the first time period; and (ii) a plot of trailing mortality, comprising a percentage of mortality of the first historical cohort for a trailing time period, the trailing time period being a windowed segment of the first time period;
2. a median survival graph, the median survival graph comprising a single graph with (i) an identification of a median survival time for the first historical cohort for the first time period; and (ii) a plot of a percentage of mortality of the first historical cohort for a pre-median survival time period; and (iii) a plot of a percentage of survival of the first historical cohort for a post-median survival time period;
3. a mortality risk graph, the mortality risk graph comprising a plot of trailing mortality, comprising a percentage of mortality, for a trailing time period, of the first historical cohort relative to a number of survivors of the first historical cohort at a beginning of the trailing time period, the trailing time period being a windowed segment of the first time period; and
4. a survival probability graph, the survival probability graph comprising a plot of survival probability over the first time period, wherein the survival probability is calculated for each of a plurality of time points over the first time period by dividing a total number of survivors of the first historical cohort at the end of the first time period by a total number of survivors of the first historical cohort at the time point being calculated.

11. A system for providing a visualization of heterogeneity of a patient's tumor and a therapy effectiveness for the patient's heterogenous tumor, comprising:
patient information comprising at least demographic and clinical information about the patient, an identification of the patient's tumor type, and a heterogeneity assessment of the patient's tumor, the heterogeneity assessment comprising an analysis of heterogeneity of the patient's tumor and an identification of two or more subclones of the tumor;
a therapy database;
a processor configured to: (i) obtain, based on the identification of the patient's tumor type and the heterogeneity assessment of the patient's tumor, therapy effectiveness information for each of the two or more subclones of the tumor from the therapy database; and (ii) generate a visual representation of the heterogeneity assessment of the patient's tumor and the obtained therapy effectiveness information for each of the two or more subclones of the tumor;
a user interface configured to provide the generated visual representation, wherein the generated visual representation comprises:
1. a tumor heterogeneity graph, comprising: (i) an identification of each of the two or more subclones of the patient's tumor, wherein the identification comprises an indication of a relative amount of each subclone in the patient's tumor; and (ii) a heterogeneity score indicating an overall complexity of tumor composition of the patient, wherein the heterogeneity score is visualized in an ordered data plot of heterogeneity scores of a same tumor type from a patient cohort, indicating a degree of tumor heterogeneity of the patient as a percentile rank within the patient cohort; and
2. a therapy effectiveness graph, comprising an indication of effectiveness of each of a plurality of different therapies for each of the two or more subclones of the patient's tumor.

12. The system of claim 11, wherein the tumor heterogeneity graph further comprises an indication of a metastatic potential of each subclone in the patient's tumor.

13. The system of claim 11, wherein the two or more subclones are differentiated by one or more molecular and/or histological variations.

14. The system of claim 11, wherein each of the two or more subclones of the patient's tumor on the tumor heterogeneity graph are selectable, and wherein selecting a subclone results in a visual display of some or all of the one or more molecular and/or histological variations specific to the selected subclone.

15. The system of claim 11, wherein the therapy effectiveness graph further comprises an indication of effectiveness of a combination of two or more therapies for the two or more subclones of the patient's tumor.
